(19) United States
(12) Patent Application Publication
(10) Pub. No.: US 2003/0166147 A1
(43) Pub. Date: Sep. 4, 2003

(54) Clonal myeloma cell lines useful for manufacturing proteins in chemically defined media

(76) Inventors: ChiChang Lee, Norristown, PA (US); Edward Savino, (US); Gordon Moore, Wayne, PA (US); Celia Ly, Lancaster, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/316,311

(22) Filed: Dec. 11, 2002

Related U.S. Application Data
(60) Provisional application No. 60/339,429, filed on Dec. 14, 2001.

(51) Int. Cl. C12N 5/06; C12N 5/08; C12P 21/02

(52) U.S. Cl. 435/69.1; 435/366; 435/326

(57) ABSTRACT

The present invention relates to clonal myeloma cell lines that have the ability to grow continuously in chemically defined media. The present invention also relates to the production of proteins in clonal myeloma cell lines and any cell lines derived therefrom. The present invention further relates to methods for identifying cell lines capable of growing in chemically defined media. The present invention also relates to business methods where customers are provided with the cells, cell lines, and cell cultures of the present invention.

### FIELD OF THE INVENTION

The present invention relates to cells, cell lines, and cell cultures useful in recombinant DNA technologies and for the production of proteins in cell culture, and further relates to clonal myeloma cell lines capable of growing in chemically defined media.

### BACKGROUND OF THE INVENTION

Traditional techniques for recombinant protein production have relied upon the use of cell culture media supplemented with chemically undefined, animal-derived components, such as serum and mixed proteins, to facilitate robust cell growth and viability. Many recombinant proteins, especially monoclonal antibodies, were employed primarily for research or in vitro diagnostic applications, leaving only limited incentive to invest time and money in the elimination of animal-derived supplements. As new technologies have developed, however, cell culture-produced proteins are becoming increasingly important as potential in vivo human therapeutic agents.

The change in the intended uses for proteins produced in cell culture has raised new concerns about the materials and methods employed for their production. For example, serum contains many components that have not been fully identified nor their role or mechanism of action determined. Thus, serum will differ from batch to batch, possibly requiring testing to determine levels of the various components and their effects on cells. In addition, serum might possibly be contaminated with microorganisms such as viruses, mycoplasma and perhaps prions, some of which may be harmless but nonetheless represent an additional unknown factor.

This sensitivity has become more acute in recent years with the emergence of Bovine Spongiform Encephalopathy (BSE), a neurodegenerative disease of cattle. Because it is transmissible to humans, the emergence of BSE has raised regulatory concerns about using animal-derived components in the production of biologically active products. Indeed, the remote possibility of contamination of the cell culture medium, and ultimately the final therapeutic drug by adventitious agents extant in animal-derived materials, has led many regulatory agencies to strongly recommend the discontinued or limited use of animal-derived materials in cell culture media.

In response to this situation, several companies have developed cell culture media for the growth and maintenance of mammalian cells that are serum-free and/or animal-derived protein-free. Unlike serum-supplemented media, which may be utilized for a broad range of cell types and culture conditions, these serum-free formulations are most often highly specific. Indeed, the multitude of commercial serum-free media formulations available demonstrates the diversity of the needs. Most media are suitable for small-scale laboratory applications but become too expensive for large-scale bioreactors. Moreover, some are appropriate for cell growth, but perform poorly as a production medium.

More recent advances in cell biology have lead to new strategies to develop cell lines or parental hosts capable of growth in chemically defined (“CD”) media. These approaches involve genetic manipulation of cellular biochemical processes including cell cycle control, apoptosis, and growth factor regulation. For example, Super CHO, Cyclin E CHOK₁, and E₂F CHOK₁ are all CHOK₁ derivatives that, as a result of various genetic manipulations, have the capability of growth and recombinant protein expression in CD media. Although promising, the practical application of such systems at the manufacturing level may limit their future use within the industry.

Consequently, there is still a great need for the development of alternative cell lines capable of manufacturing recombinant proteins at large scale, commercial capacity while growing in CD media.

### SUMMARY OF THE INVENTION

The present invention relates to cells, cell lines, and cell cultures useful in recombinant DNA technologies and for the production of proteins in cell culture. Specifically, the present invention relates to clonal myeloma cell lines or any cell lines derived therefrom that are capable of growing continuously in a chemically defined medium; growing to high cell density in a chemically defined medium; remaining viable after cryopreservation in the absence of serum; and detectably expressing recombinant proteins following genetic manipulation and/or subsequent culture in a chemically defined medium.

In a preferred embodiment, the expression of proteins is accomplished by manipulating the cells, cell lines, and cell cultures to express at least one protein in detectable amount. The manipulation step may be accomplished by introducing a nucleic acid encoding at least one protein into the cells, cell lines, and cell cultures of the present invention. The nucleic acid encoding at least one protein may be introduced by one of several methods including, but not limited to, electroporation, lipofection, calcium phosphate precipitation, polyethylene glycol precipitation, sonication, transfection, transduction, transformation, and viral infection.

In an alternative embodiment, the cells, cell lines, and cell cultures of the present invention are manipulated to express at least one desired protein in detectable amounts by inducing transcription and translation of a nucleic acid encoding at least one protein when such nucleic acid already exists in the cells, cell lines, and cell cultures.

In a preferred embodiment, the protein expressed in the cells, cell lines, and cell cultures of the present invention is a diagnostic protein. Alternatively, the protein may be a therapeutic protein. The diagnostic or therapeutic protein may be an immunoglobulin, a cytokine, an integrin, an antigen, a growth factor, a receptor or fusion protein thereof, any fragment thereof, or any structural or functional analog thereof. The diagnostic or therapeutic protein may also be a cell cycle protein, a hormone, a neurotransmitter, a blood protein, an antimicrobial, a receptor or fusion protein thereof, any fragment thereof, or any structural or functional analog thereof.

In a preferred embodiment, the cells, cell lines, and cell cultures of the present invention may produce an immunoglobulin or fragment thereof derived from a rodent or a primate. More specficially, the immunoglobulin or fragment thereof may be derived from a mouse or a human. Alternatively, the immunoglobulin or fragment thereof may be chimeric or engineered. Indeed, the present invention further contemplates cells, cell lines, and cell cultures that produce an immunoglobulin or fragment thereof which is humanized, CDR grafted, phage displayed, transgenic mouse-produced, optimized, mutagenized, randomized or recombined.

The cells, cell lines, and cell cultures of the present invention may produce an immunoglobulin or fragment thereof including, but not limited to, IgG1, IgG2, IgG3, IgG4, IgA1, IgA2, slgA, IgD, IgE, and any structural or functional analog thereof. In a specific embodiment, the immunoglobulin expressed in the cells, cell lines, and cell cultures of the present invention is infliximab. Alternatively, the immunoglobulin may be rTNV148B.

Furthermore, the immunoglobulin fragment produced by the cells, cell lines, and cell cultures of the present invention may include, but is not limited to, F(ab′)₂, Fab′, Fab, Fc, Facb, pFc′, Fd, Fv, and any structural or functional analog thereof. In a specific embodiment, the immunoglobulin fragment is abciximab.

The present invention further provides cells, cell lines, and cell cultures that express an immunoglobulin or fragment thereof which binds an antigen, a cytokine, an integrin, an antigen, a growth factor, a cell cycle protein, a hormone, a neurotransmitter, a receptor or fusion protein thereof, a blood protein, an antimicrobial, any fragment thereof, and any structural or functional analog of any of the foregoing.

In one embodiment of the present invention, the cells, cell lines, and cell cultures produce an integrin. Examples of integrins contemplated by the present invention include, but are not limited to, α1, α2, α3, α4, α5, α6, α7, α8, α9, αD, αL, αM, αV, αX, αIIb, αIELb, β1, β2, β3, β4, β5, β6, β7, β8, α1β1, α2β1, α3β1, α4β1, α5β1, α6β1, α7β1, α8β1, α9β1, α4β7, α6β4, αDβ2, αLβ2, αMβ2, αVβ1, αVβ3, αVβ5, αVβ6, αVβ8, αXβ2, αIIbβ3, αIELbβ7, and any structural or functional analog thereof.

In an embodiment of the invention, the recombinant protein expressed by the cells, cell lines, and cell cultures of the present invention is an antigen. The antigen may be derived from a number of sources including, but not limited to, a bacterium, a virus, a blood protein, a cancer cell marker, a prion, a fungus, and any structural or functional analog thereof.

In yet another embodiment, the cells, cell lines, and cell cultures of the present invention may detectably express a growth factor. Examples of the growth factors contemplated by the present invention include, but are not limited to, a human growth factor, a platelet derived growth factor, an epidermal growth factor, a fibroblast growth factor, a nerve growth factor, a human chorionic gonadotropin, an erythrpoeitin, an activin, an inhibin, a bone morphogenic protein, a transforming growth factor, an insulin-like growth factor, and any structural or functional analog thereof.

In an alternative embodiment, the cells, cell lines, and cell cultures of the present invention produce a recombinant cell cycle protein. Such cell cycle proteins include, but are not limited to, a cyclin, a cyclin-dependent kinase, a tumor suppressor gene, a caspase protein, a Bcl-2, a p70 S6 kinase, an anaphase-promoting complex, a S-phase promoting factor, a M-phase promoting factor, and any structural or functional analog thereof.

The present invention further provides cells, cell lines, and cell cultures that express a cytokine. Examples of cytokines contemplated by the present invention include, but are not limited to, an interleukin, an interferon, a colony stimulating factor, a tumor necrosis factor, an adhesion molecule, an angiogenin, an annexin, a chemokine, and any structural or functional analog thereof.

In another embodiment, the recombinant protein expressed by the cells, cell lines, and cell cultures of the present invention is a growth hormone. The growth hormone may include, but is not limited to, a human growth hormone, a growth hormone, a prolactin, a follicle stimulating hormone, a human chorionic gonadotrophin, a leuteinizing hormone, a thyroid stimulating hormone, a parathyroid hormone, an estrogen, a progesterone, a testosterone, an insulin, a proinsulin, and any structural or functional analog thereof.

The present invention further relates to the expression of neurotransmitters using the cells, cell lines, and cell cultures taught herein. Examples of neurotransmitters include, but are not limited to, an endorphin, a coricotropin releasing hormone, an adrenocorticotropic hormone, a vaseopressin, a giractide, a N-acytlaspartylglutamate, a peptide neurotransmitter derived from pre-opiomelanocortin, any antagonists thereof, and any agonists thereof.

In another embodiment, the cells, cell lines, and cell cultures of the present invention are manipulated to produce a receptor or fusion protein. The receptor or fusion protein may be, but is not limited to, an interleukin-1, an interleukin-12, a tumor necrosis factor, an erythropoeitin, a tissue plasminogen activator, a thrombopoetin, and any structural or functional analog thereof.

Alternatively, recombinant blood proteins may be expressed in the cells, cell lines, and cell cultures of the present invention. Such recombinant proteins include, but are not limited to, an erythropoeitin, a thrombopoeitin, a tissue plasminogen activator, a fibrinogen, a hemoglobin, a transferrin, an albumin, a protein c, and any structural or functional analog thereof. In a specific embodiment, the cells, cell lines, and cell cultures of the present invention express tissue plasminogen activator.

In another embodiment, the cells, cell lines and cell cultures of the present invention produce a recombinant antimicrobial agent. Examples of antimicrobial agents contemplated by the present invention include, for example, a beta-lactam, an aminoglycoside, a polypeptide antibiotic, and any structural or functional analog thereof.

In a preferred embodiment, the cells, cell lines, and cell cultures of the present invention produce recombinant proteins at about 0.01 mg/L to about 10,000 mg/L of culture medium. In another embodiment, the cells, cell lines, and cell cultures of the present invention produce recombinant proteins at a level of about 0.1 pg/cell/day to about 100 ng/cell/day.

The present invention further provides methods for producing at least one protein from a cultured cell. In a preferred embodiment, cells of the present invention that express at least one desired protein are cultured in a chemically defined medium and the proteins are isolated from the chemically defined medium or from the cells themselves. In addition, the present invention further relates to recombinant proteins obtained by this method.

The present invention also provides methods for identifying cell lines capable of growing continuously in a chemically defined medium. In a preferred embodiment, cells from one type of cell line, which are not known to grow in a chemically defined medium, are cultured in the chemically defined medium and spontaneous mutant cells that are capable of growing in the chemically defined medium are selected. Moreover, the present invention relates to at least one cell line obtained according to this method.

The present invention further relates to business methods where the cells, cell lines, cell cultures, and recombinant proteins obtained therefrom are provided to customers. In a specific embodiment, a customer is provided with a cell line of the present invention. In another embodiment, a customer is provided with a recombinant protein derived from a cell line of the present invention.

### BRIEF DESCRIPTION OF THE DRAWINGS

FIG. 1a depicts cell line C463A post-thaw viability at 0 hours and 24 hours. FIG. 1b is a graph depicting growth profiles of C463A grown in both Sigma® Serum and Protein-Free Medium (a CD medium) and CD-Hybridoma medium (a CD medium) following freeze/thaw in CD-Hybridoma medium with 10% DMSO. FIG. 1b shows the results of a growth profile of Sp_{2/0} parental cells grown in CD-Hybridoma medium following freeze/thaw in IMDM, 20% FBS.

FIG. 2 is a graph showing the growth profile of C463A semi-batch culture in CD-Hyrbidoma medium versus the growth profile of Sp_{2/0} semi-batch culture in CD-Hybridoma medium. Total (TC) and viable cell (VC) densities are indicated.

FIG. 3 is a graph illustrating the growth profile of C463A semi-batch culture in CD-Hybridoma medium versus the growth profile of Sp_{2/0} semi-batch culture in IMDM, 5% FBS (a chemically undefined medium). Total cell (TC) and viable cell (VC) densities for days 3-7 are indicated.

FIG. 4 presents four graphs that illustrate the growth profiles of cell line C524A in both IMDM, 5% FBS and CD-Hybridoma medium versus the growth profile of C466D in IMDM, 5% FBS. FIG. 4a depicts the percent viability over time for cells grown in spinner flasks. FIG. 4b illustrates viable cell density over time of cells grown in spinner flasks. FIG. 4c shows total cell density over time of cells grown in spinner flasks. FIG. 4d portrays IgG titer over time for cells grown in spinner flasks.

FIG. 5 contains four graphs that compare the growth profile of C524A in CDM medium and CD-Hybridoma medium, both of which are CD media. FIG. 5a illustrates the percent viability over time for cells grown in spinner flasks. FIG. 5b shows viable cell density over time of cells grown in spinner flasks. FIG. 5c portrays total cell density over time of cells grown in spinner flasks. FIG. 5d depicts IgG titer over time for cells grown in spinner flasks.

FIG. 6 presents four graphs that represent data generated during an 11-passage stability study of C524A grown in both CDM medium and CD-Hybridoma medium. FIG. 6a shows the percent viability over time for cells grown in spinner flasks. FIG. 6b portrays mean doubling times over time of cells grown in spinner flasks. FIG. 6c depicts total cell density over time of cells grown in spinner flasks. FIG. 6d illustrates IgG titer over time for cells grown in spinner flasks.

FIG. 7 contains four graphs that compare the growth profile of C524A in CDM medium with the growth profile of C524A in CD-Hybridoma medium after an 11-passage stability study. FIG. 7a portrays the percent viability over time for cells grown in spinner flasks. FIG. 7b depicts viable cell density over time of cells grown in spinner flasks. FIG. 7c illustrates total cell density over time of cells grown in spinner flasks. FIG. 7d shows IgG titer over time for cells grown in spinner flasks.

### DETAILED DESCRIPTION OF THE INVENTION

It is to be understood that this invention is not limited to the particular methodology, protocols, cell lines, animal species or genera, constructs, and reagents described and as such may vary. It is also to be understood that the terminology used herein is for the purpose of describing particular embodiments only, and is not intended to limit the scope of the present invention.

It must be noted that as used herein and in the appended claims, the singular forms “a,” “and,” and “the” include plural reference unless the context clearly dictates otherwise. Thus, for example, reference to “a protein” is a reference to one or more proteins and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

Accordingly, the present invention relates to clonal myeloma cell lines that have the ability to grow continuously in CD media. These clonal myeloma cell lines may derived from any number of commercially available myeloma cell lines, including, but not limited to, Sp_{2/0}-Ag14 (American Type Culture Collection (“ATCC”), Manassas, Va., ATCC CRL No. 1851); P3X63Ag8.653 (ATCC CRL No. 1580); RPMI 8226 (ATCC CRL No. 155); and NSO (European Collection of Cell Cultures (“ECACC”), Salisbury, Wiltshire, U.K., ECACC No. 85110503). Other myeloma cell lines are available from cell culture depositories such as ATCC; ECACC; Istituto Zooprofilattico Sperimentale (“IZSBS”), Brescia, Italy; Human and Animal Cell Cultures (“DSMZ”), Braunschweig, F. R. G.; and Interlab Cell Line Collection (“ICLC”), Genova, Italy.

In one embodiment, the clonal myeloma cell line is a spontaneous mutant cloned from a Sp_{2/0}-Ag14 (“Sp_{2/0}”) cell bank in CD media. In this embodiment, the clonal myeloma cell line is designated C463A. Characterization of C463A revealed that the cell line has a number of unique growth characteristics not associated with parental Sp_{2/0} cells. For example, C463A may be frozen and thawed in the absence of serum, a necessary cryopreservation agent for Sp_{2/0} parental cell lines. In addition, unlike parental lines, C463A can grow to high cell density in CD media. Further characterization demonstrated that C463A grown in CD media exhibits growth parameters, including viable cell density and doubling time, that are similar or superior to those observed when cells are maintained in growth medium supplemented with serum.

CD media, as used in the present invention, comprises growth media that are devoid of any components of animal origin, including serum, serum proteins, hydrolysates, or compounds of unknown composition. All components of CD media have a known chemical structure, resulting in the elimination of the batch-to-batch variability discussed previously. The CD media used in the present invention may include, but is not limited to, CD-Hybridoma, a CD medium produced by Invitrogen Corp., Carlsbad, Calif. (Cat. No. 11279-023). For growth profiles, CD-Hybridoma medium was supplemented with 1 g/L NaHCO₃ and L-Glutamine to final concentration of 6 mM. The present invention also contemplates the use of the chemically defined media, including “CDM medium,” described in Centocor's pending patent application, Serial No. 60/268,849, entitled “Chemically Defined Medium For Cultured Mammalian Cells,” which is expressly incorporated by reference.

In contrast to CD media, protein-free media may still contain components of animal origin (e.g., cystine extracted from human hair) and/or undefined components of animal or plant origin (e.g., various hydrolysates which contribute low molecular weight peptides). Protein-free media are a step closer to a defined formulation than serum-free media, which may contain discrete proteins or bulk protein fractions. Notably, growth medium that is both serum-free and protein-free may be, in effect, a CD medium. Indeed, the present invention further contemplates the growth of C463A in Sigma® Serum and Protein-Free medium (Cat. No. S-8284), Sigma-Aldrich Corp., St. Louis, Mo., supplemented with 8 mM L-Glutamine for growth profiles.

As stated above, the present invention comprises a spontaneous mutant derived from the myeloma cell line Sp_{2/0}. Briefly, Sp_{2/0} cells were seeded at a density of 40 cells/well in five 9 well cluster dishes with Sigma® Serum and Protein-Free Medium. Fourteen days after subcloning in Sigma® Serum and Protein-Free Medium, 37 wells (seven percent) contained viable colonies. Twenty of the thirty-seven colonies were expanded in 6-well plates. Five primary candidate lines were visually identified and growth profiles at the T-75 stage were initiated. Three secondary candidate cell lines were expanded and the remaining lines were pooled and frozen. Of the three secondary candidate cell lines, the clone designated 2D11 was the most successful cell line, as indicated by its growth profile, and this line was subsequently designated C463A. C463A was further expanded and analyzed for its ability to grow in various CD media.

Analysis of the cell line of the present invention revealed that C463A has the ability to sustain continuous growth in CD media. C463A cultures were established in CD media (both CD-Hybridoma medium and Sigma® Serum and Protein-Free medium), routine maintenance performed (cell cultures split three times per week) and various growth parameters recorded. Table 1 shows the averages for several cell growth parameters over the course of ten consecutive passages (one month).

TABLE 1
C463A continuous culture in CD media

| | | | | Doubling |
|---|---|---|---|---|
| | | Total Density | Percent | Time |
| Cell Line | Medium | (10⁶ Cell/ml) | Viability | (Hrs) |
| C463A | CD-Hybridoma | 1.35 | 93% | 20 |
| C463A | Sigma ® Serum | 0.94 | 91% | 21 |
| | and Protein-Free | | | |
| Sp_{2/0} | IMDM, 5% FBS | 1.7 | 95% | 18 |

In both types of CD media tested, C463A reached a total cell density comparable to that of Sp_{2/0} parental cells grown in Iscove's Modified Dulbecco's Medium (IMDM), 5% Fetal Bovine Serum (FBS) (optimal medium). In addition, the percent viability and doubling time of C463A grown in CD media were also similar to that observed for Sp_{2/0} parental cells grown in optimal medium.

Further characterization of C463A indicated that the cell line has a number of unique growth characteristics not associated with the Sp_{2/0} parental cells. For example, fetal bovine serum is not necessary when freezing, thawing, and establishing C463A culture. Briefly, C463A cells were grown to exponential growth phase in T-flasks or spinners. After spinning the cells at 800-1000 rpm, the cells were resuspended in 5 ml of CD-Hybridoma medium supplemented with 10% Dimethyl Sulfoxide (DMSO) at a density of 1×10⁷ vc/ml (viable cells/ml). One milliliter aliquots were placed in cryovials and frozen overnight at −70° C. The vials were transferred to liquid nitrogen vapor phase within one week for long-term storage. After thawing in CD-Hybridoma medium, cell viabilities were measured at 0 and 24 hours, and cultures established in CD-Hybridoma medium.

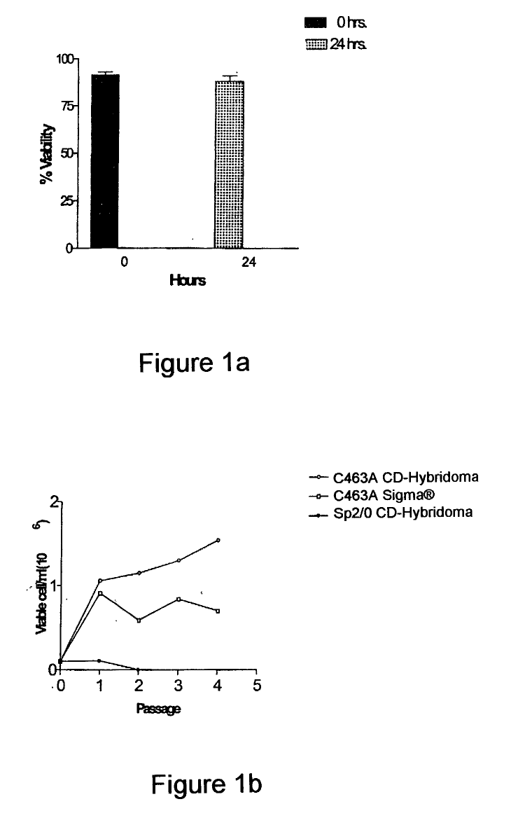

Referring to FIG. 1, FIG. 1a indicates that post-thaw viabilities of C463A ranged between eighty-five to ninety percent, which is identical to Sp_{2/0} parental cells when frozen in the presence of 20% FBS (eight-five to ninety percent, data not shown). FIG. 1b indicates that growth profiles of C463A cultures established in both Sigma® Serum and Protein-Free medium and CD-Hybridoma medium were typical in continuous culture conditions. Sp_{2/0} parental cells, however, grew poorly and were discontinued after the second passage in CD-Hybridoma medium.

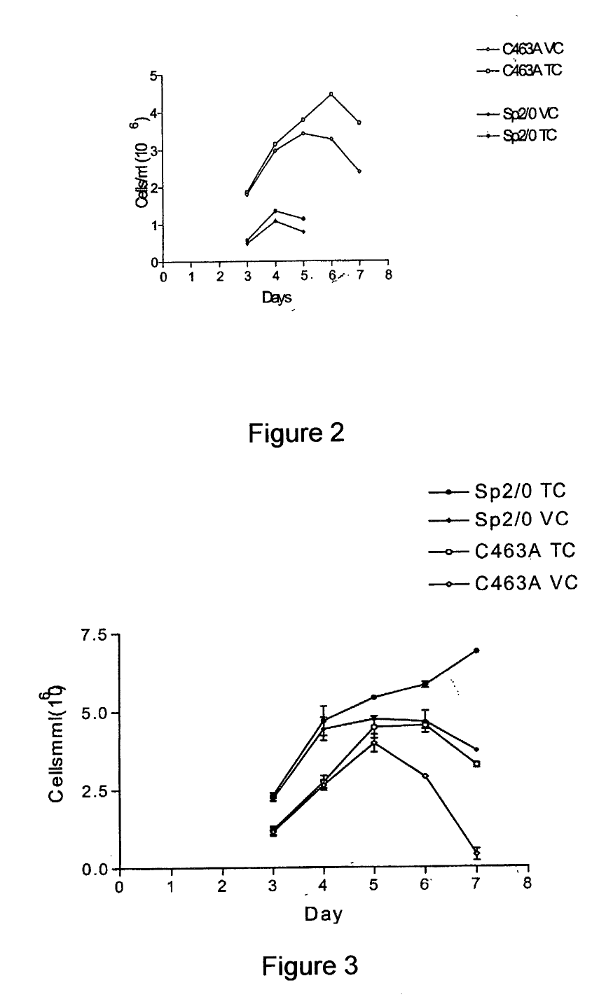

Another unique characteristic of C463A is its ability to achieve high cell density in CD media. FIG. 2 illustrates the growth profiles of C463A semi-batch culture in CD-Hybridoma medium versus the growth profile of Sp_{2/0} semi-batch culture in CD-Hybridoma medium. Semi-batch cultures provide the advantage of accumulating cells to high density by manually removing old medium and recycling total cells. Briefly, a semi-batch growth profile (seventy-five percent media changed daily 3 days post-inoculation) was initiated in CD-Hybridoma medium and growth parameters examined daily (days 3-7). As shown in FIG. 2, where “VC” means viable cells/ml (10⁶) and “TC” means total cells/ml (10⁶), C463A growth and viability exceeded Sp_{2/0} parental cells in the conditions described. Viable and total cell densities of 3.27×10⁶ vc/ml and 4.45×10⁶ cells/ml were observed on day six for C463A, while control numbers were significantly less at 1×10⁶ vc/ml and 1.35×10⁶ cells/ml on day four.

To create a more stringent positive control to evaluate C463A growth in CD semi-batch conditions, the experiment described above was repeated and compared with Sp_{2/0} parental cells grown in IMDM, 5% FBS. The data shown in FIG. 3 indicate that C463A achieved cell densities comparable to Sp_{2/0} parental cells. C463A viable and total cell densities of 3.75×10⁶ vc/ml and 4.25×10⁶ cells/ml were observed on day five, while Sp_{2/0} parental cells grew to viable and total cell densities of 4.75×10⁶ vc/ml and 5.5×10⁶ cells/ml over the same period. In addition, cell culture viability was identical (eighty-nine percent, data not shown) on day five and doubling times (days 3-5, data not shown) were 19 and 21 hours for Sp_{2/0} and C463A, respectively. This experiment demonstrates that C463A can achieve cell density in CD media that is equal or superior to Sp_{2/0} parental cells cultured in optimal growth media.

The experiments described above demonstrate the ability of C463A to grow in CD media at least as well as Sp_{2/0} parental cells in optimal media. More importantly C463A may be manipulated to stably express recombinant proteins. In one embodiment, cell line C463A is manipulated to produce recombinant proteins at a level of about 0.01 mg/L to about 10,000 mg/L of culture medium. In another embodiment, cell line C463A is manipulated to produce recombinant proteins at a level of about 0.1 pg/cell/day to about 100 ng/cell/day.

The present invention further relates to other clonal myeloma cell lines that have the ability to grow in CD media. Such cell lines may be manipulated to stably express recombinant proteins by using methods known in the art or as taught herein. For example, the clonal myeloma cell lines of the present invention may be manipulated to produce recombinant proteins at a level of about 0.01 mg/L to about 10,000 mg/L of culture medium. In another embodiment, the clonal myeloma cell lines of the present invention may be manipulated to produce recombinant proteins at a level of about 0.1 pg/cell/day to about 100 ng/cell/day.

The introduction of nucleic acids encoding recombinant proteins may be accomplished via any one of a number of techniques well known in the art, including, but not limited to, electroporation, lipofection, calcium phosphate precipitation, polyethylene glycol precipitation, sonication, transfection, transduction, transformation, and viral infection. Indeed, molecular techniques are well known in the art. See Sambrook et al Molecular Cloning: A Lab. Manual (2001); Ausbel et al., Current Protocols in Molecular Biology (1995).

A variety of mammalian expression vectors may be used to express recombinant proteins in the cell culture taught herein. Commercially available mammalian expression vectors that may be suitable for recombinant protein expression include, but are not limited to, pMAMneo (Clontech, Palo Alto, Calif.), pcDNA3 (Invitrogen, Carlsbad, Calif.), pMClneo (Stratagene, La Jolla, Calif.), pXTI (Stratagene, La Jolla, Calif.), pSG5 (Stratagene, La Jolla, Calif.), EBO-pSV2-neo (American Type Culture Collection (“ATCC”), Manassas, Va., ATCC No. 37593), pBPV-1(8-2) (ATCC No. 37110), pdBPV-MMTneo(342-12) (ATCC No. 37224), pRSVgpt (ATCC No. 37199), pRSVneo (ATCC No. 37198), pSV2-dhfr (ATCC No. 37146), pUCTag (ATCC No. 37460), and 17D35 (ATCC No. 37565).

The cells, cell lines, and cell cultures of the present invention may be used as a suitable hosts for a variety of recombinant proteins. Such proteins include immunoglobulins, integrins, antigens, growth factors, cell cycle proteins, cytokines, hormones, neurotransmitters, receptor or fusion proteins thereof, blood proteins, antimicrobials, or fragments, or structural or functional analogs thereof. These following descriptions do not serve to limit the scope of the invention, but rather illustrate the breadth of the invention.

For example, in one embodiment of the invention, the immunoglobulin may be derived from human or non-human polyclonal or monoclonal antibodies. Specifically, these immunoglobulins (antibodies) may be recombinant and/or synthetic human, primate, rodent, mammalian, chimeric, humanized or CDR-grafted, antibodies and anti-idiotype antibodies thereto. These antibodies can also be produced in a variety of truncated forms in which various portions of antibodies are joined together using genetic engineering techniques. As used presently, an “antibody,” “antibody fragment,” “antibody variant,” “Fab,” and the like, include any protein- or peptide-containing molecule that comprises at least a portion of an immunoglobulin molecule, such as but not limited to at least one CDR of a heavy or light chain or a ligand binding portion thereof, a heavy chain or light chain variable region, a heavy chain or light chain constant region, a framework region, or any portion thereof, which may be expressed in the cell culture of the present invention. Such antibodies optionally further affect a specific ligand, such as but not limited to, where such antibody modulates, decreases, increases, antagonizes, agonizes, mitigates, alleviates, blocks, inhibits, abrogates and/or interferes with at least one target activity or binding, or with receptor activity or binding, in vitro, in situ and/or in vivo.

In one embodiment of the invention, such antibodies, or functional equivalents thereof, may be “human,” such that they are substantially non-immunogenic in humans. These antibodies may be prepared through any of the methodologies described herein, including the use of transgenic animals, genetically engineered to express human antibody genes. For example, immunized transgenic mice (xenomice) that express either fully human antibodies, or human variable regions have been described. See WO 96/34096. In the case of xenomice, the antibodies produced include fully human antibodies and can be obtained from the animal directly (e.g., from serum), or from immortalized B-cells derived from the animal, or from the genes encoding the immunoglobulins with human variable regions can be recovered and expressed to obtain the antibodies directly or modified to obtain analogs of antibodies such as, for example, Fab or single chain Fv molecules. Id. These genes are then introduced into the cells, cell lines, and cell cultures of the present invention by methods known in the art, or as taught herein.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof, that are expressed in the cell culture of the present invention. The present invention thus encompasses antibody fragments capable of binding to a biological molecule (such as an antigen or receptor) or portions thereof, including but not limited to Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments. See, e.g., Current Protocols in Immunology, (Colligan et al., eds., John Wiley & Sons, Inc., N.Y., 1994-2001).

As with antibodies, other peptides that bind a particular target protein or other biological molecule (target-binding peptides) may be produced by the cells, cell lines, and cell cultures disclosed herein. Such target-binding peptides may be isolated from tissues and purified to homogeneity, or isolated from cells that contain the target-binding protein, and purified to homogeneity. Once isolated and purified, such target-binding peptides may be sequenced by well-known methods. From these amino acid sequences, DNA probes may be produced and used to obtain mRNA, from which cDNA can be made and cloned by known methods. Other well-known methods for producing cDNA are known in the art and may effectively be used. In general, any desired peptide can be isolated from any cell or tissue expressing such proteins using a cDNA probe such as the probe described above, isolating mRNA and transcribing the mRNA into cDNA. Thereafter, the protein can be produced by inserting the cDNA into an expression vector, such as a virus, plasmid, cosmid, or other vector, inserting the expression vector into a cell, proliferating the resulting cells, and isolating the expressed target-binding protein from the medium or from cell extract as described above. See, e.g., U.S. Pat. No. 5,808,029.

Alternatively, recombinant peptides, including antibodies, may be identified using various library screening techniques. For example, peptide library screening takes advantage of the fact that molecules of only “peptide” length (2 to 40 amino acids) can bind to the receptor protein of a given large protein ligand. Such peptides may mimic the bioactivity of the large protein ligand (“peptide agonists”) or, through competitive binding, inhibit the bioactivity of the large protein ligand (“peptide antagonists”). Phage display peptide libraries have emerged as a powerful method in identifying such peptide agonists and antagonists. In such libraries, random peptide sequences are displayed by fusion with coat proteins of filamentous phage. Typically, the displayed peptides are affinity-eluted against an immobilized extracellular domain of an antigen or receptor. The retained phages may be enriched by successive rounds of affinity purification and repropagation. The best binding peptides may be sequenced to identify key residues within one or more structurally related families of peptides. The peptide sequences may also suggest which residues may be safely replaced by alanine scanning or by mutagenesis at the DNA level. Mutagenesis libraries may be created and screened to further optimize the sequence of the best binders. See, e.g., WO 00/24782; WO 93/06213; U.S. Pat. No. 6,090,382.

Other display library screening method are known as well. For example, E. coli displays employ a peptide library fused to either the carboxyl terminus of the lac-repressor or the peptidoglycan-associated lipoprotein, and expressed in E. coli. Ribosome display involves halting the translation of random RNAs prior to ribosome release, resulting in a library of polypeptides with their associated RNAs still attached. RNA-peptide screening employs chemical linkage of peptides to RNA. Additionally, chemically derived peptide libraries have been developed in which peptides are immobilized on stable, non-biological materials, such as polyethylene rods or solvent-permeable resins. Another chemically derived peptide library uses photolithography to scan peptides immobilized on glass slides. These methods of chemical-peptide screening may be advantageous because they allow use of D-amino acids and other unnatural analogues, as well as non-peptide elements. See WO 00/24782.

Moreover, structural analysis of protein-protein interaction may also be used to suggest peptides that mimic the binding activity of large protein ligands. In such an analysis, the crystal structure may suggest the identity and relative orientation of critical residues of the large protein ligand, from which a peptide may be designed. These analytical methods may also be used to investigate the interaction between a receptor protein and peptides selected by phage display, which may suggest further modification of the peptides to increase binding affinity. Thus, conceptually, one may discover peptide mimetics of any protein using phage display and the other methods mentioned above. For example, these methods provide for epitope mapping, for identification of critical amino acids in protein-protein interactions, and as leads for the discovery of new therapeutic agents. See WO 00/24782.

The nature and source of the recombinant protein expressed in the cells, cell lines, and cell cultures of the present invention is not limited. The following is a general discussion of the variety of proteins, peptides and biological molecules that may be used in the in accordance with the teachings herein. These descriptions do not serve to limit the scope of the invention, but rather illustrate the breadth of the invention.

Thus, an embodiment of the present invention may include the production of one or more growth factors. Briefly, growth factors are hormones or cytokine proteins that bind to receptors on the cell surface, with the primary result of activating cellular proliferation and/or differentiation. Many growth factors are quite versatile, stimulating cellular division in numerous different cell types; while others are specific to a particular cell-type. The following Table 2 presents several factors, but is not intended to be comprehensive or complete, yet introduces some of the more commonly known factors and their principal activities.

TABLE 2
Growth Factors

| Factor | Principal Source | Primary Activity | Comments |
|---|---|---|---|
| Platelet Derived | Platelets, endothelial | Promotes proliferation of | Dimer required for |
| Growth Factor | cells, placenta. | connective tissue, glial and | receptor binding. |
| (PDGF) | | smooth muscle cells. PDGF | Two different protein |
| | | receptor has intrinsic tyrosine | chains, A and B, form |
| | | kinase activity. | 3 distinct dimer |
| | | | forms. |
| Epidermal | Submaxillary gland, | promotes proliferation of | EGF receptor has |
| Growth Factor | Brunners gland. | mesenchymal, glial and | tyrosine kinase |
| (EGF) | | epithelial cells | activity, activated in |
| | | | response to EGF |
| | | | binding. |
| Fibroblast | Wide range of cells; | Promotes proliferation of | Four distinct |
| Growth Factor | protein is associated with | many cells including skeletal | receptors, all with |
| (FGF) | the ECM; nineteen family | and nervous system; inhibits | tyrosine kinase |
| | members. Receptors | some stem cells; induces | activity. FGF |
| | widely distributed in | mesodermal differentiation. | implicated in mouse |
| | bone, implicated in | Non-proliferative effects | mammary tumors and |
| | several bone-related | include regulation of pituitary | Kaposi's sarcoma. |
| | diseases. | and ovarian cell function. | |
| NGF | | Promotes neurite outgrowth | Several related |
| | | and neural cell survival | proteins first |
| | | | identified as protooncogenes; |
| | | | trkA |
| | | | (trackA), trkB, trkC |
| Erythropoietin | Kidney | Promotes proliferation and | Also considered a |
| (Epo) | | differentiation of erythrocytes | ‘blood protein,’ and a |
| | | | colony stimulating |
| | | | factor. |
| Transforming | Common in transformed | Potent keratinocyte growth | Related to EGF. |
| Growth Factor a | cells, found in | factor. | |
| (TGF-a) | macrophages and | | |
| | keratinocytes | | |
| Transforming | Tumor cells, activated | Anti-inflammatory (suppresses | Large family of |
| Growth Factor v | TH₁ cells (T-helper) and | cytokine production and class | proteins including |
| (TGF-b) | natural killer (NK) cells | II MHC expression), | activin, inhibin and |
| | | proliferative effects on many | bone morpho-genetic |
| | | mesenchymal and epithelial | protein. Several |
| | | cell types, may inhibit | classes and |
| | | macrophage and lymphocyte | subclasses of cell- |
| | | proliferation. | surface receptors |
| Insulin-Like | Primarily liver, produced | Promotes proliferation of | Related to IGF-II and |
| Growth Factor-I | in response to GH and | many cell types, autocrine and | proinsulin, also called |
| (IGF-I) | then induces subsequent | paracrine activities in addition | Somatomedin C. |
| | cellular activities, | to the initially observed | IGF-I receptor, like |
| | particularly on bone | endocrine activities on bone. | the insulin receptor, |
| | growth | | has intrinsic tyrosine |
| | | | kinase activity. IGF-I |
| | | | can bind to the |
| | | | insulin receptor. |
| Insulin-Like | Expressed almost | Promotes proliferation of | IGF-II receptor is |
| Growth | exclusively in embryonic | many cell types primarily of | identical to the |
| Factor-II | and neonatal tissues. | fetal origin. Related to IGF-I | mannose-6-phosphate |
| (IGE-II) | | and proinsulin. | receptor that is |
| | | | responsible for the |
| | | | integration of |
| | | | lysosomal enzymes |

Additional growth factors that may be produced in accordance with the present invention include insulin and proinsulin (U.S. Pat. No. 4,431,740); Activin (Vale et al., 321 Nature 776 (1986); Ling et al., 321 Nature 779 (1986)); Inhibin (U.S. Pat. Nos. 4,740,587; 4,737,578); and Bone Morphongenic Proteins (BMPs) (U.S. Pat. No. 5,846,931; Wozney, Celluar & Molecular Biology of Bone 131-167 (1993)).

In addition to the growth factors discussed above, the present invention may be useful for the production of other cytokines. Secreted primarily from leukocytes, cytokines stimulate both the humoral and cellular immune responses, as well as the activation of phagocytic cells. Cytokines that are secreted from lymphocytes are termed lymphokines, whereas those secreted by monocytes or macrophages are termed monokines. A large family of cytokines are produced by various cells of the body. Many of the lymphokines are also known as interleukins (ILs), since they are not only secreted by leukocytes but also able to affect the cellular responses of leukocytes. Specifically, interleukins are growth factors targeted to cells of hematopoietic origin. The list of identified interleukins grows continuously. See, e.g., U.S. Pat. Nos. 6,174,995, 6,143,289; Sallusto et al., 18 Annu. Rev. Immunol. 593 (2000); Kunkel et al., 59 J. Leukocyte Biol. 81 (1996).

Additional growth factor/cytokines encompassed in the present invention include pituitary hormones such as human growth hormone (HGH), follicle stimulating hormones (FSH, FSH α, and FSH β), Human Chorionic Gonadotrophins (HCG, HCG α, HCG β), uFSH (urofollitropin), Gonatropin releasing hormone (GRH), Growth Hormone (GH), leuteinizing hormones (LH, LH α, LH β), somatostatin, prolactin, thyrotropin (TSH, TSH α, TSH β), thyrotropin releasing hormone (TRH), parathyroid hormones, estrogens, progesterones, testosterones, or structural or functional analog thereof. All of these proteins and peptides are known in the art.

The cytokine family also includes tumor necrosis factors, colony stimulating factors, and interferons. See, e.g., Cosman, 7 Blood Cell Biochem. (Whetten et al., eds., Plenum Press, New York, 1996); Gruss et al., 85 Blood 3378 (1995); Beutler et al., 7 Annu. Rev. Immunol. 625 (1989); Aggarwal et al., 260 J. Biol. Chem. 2345 (1985); Pennica et al., 312 Nature 724 (1984); R & D Systems, Cytokine Mini-Reviews, at http://www.rndsystems.com.

Several cytokines are introduced, briefly, in Table 3 below.

TABLE 3
Cytokines

| Cytokine | Principal Source | Primary Activity |
|---|---|---|
| Interleukins | Primarily macrophages but also | Costimulation of APCs and T cells; |
| IL1-a and -b | neutrophils, endothelial cells, smooth | stimulates IL-2 receptor production and |
| | muscle cells, glial cells, astrocytes, B- | expression of interferon-γ; may induce |
| | and T-cells, fibroblasts, and | proliferation in non-lymphoid cells. |
| | keratinocytes. | |
| IL-2 | CD4+ T-helper cells, activated TH₁ | Major interleukin responsible for clonal |
| | cells, NK cells. | T-cell proliferation. IL-2 also exerts |
| | | effects on B-cells, macrophages, and |
| | | natural killer (NK) cells. IL-2 receptor is |
| | | not expressed on the surface of resting T- |
| | | cells, but expressed constitutively on NK |
| | | cells, that will secrete TNF-a, IFN-g and |
| | | GM-CSF in response to IL-2, which in |
| | | turn activate macrophages. |
| IL-3 | Primarily T-cells | Also known as multi-CSF, as it stimulates |
| | | stem cells to produce all forms of |
| | | hematopoietic cells. |
| IL-4 | TH₂ and mast cells | B cell proliferation, eosinophil and mast |
| | | cell growth and function, IgE and class II |
| | | MHC expression on B cells, inhibition of |
| | | monokine production |
| IL-5 | TH₂ and mast cells | eosinophil growth and function |
| IL-6 | Macrophages, fibroblasts, endothelial | IL-6 acts in synergy with IL-1 and TNF-α |
| | cells and activated T-helper cells. | in many immune responses, including T- |
| | Does not induce cytokine expression. | cell activation; primary inducer of the |
| | | acute-phase response in liver; enhances |
| | | the differentiation of B-cells and their |
| | | consequent production of |
| | | immunoglobulin; enhances |
| | | Glucocorticoid synthesis. |
| IL-7 | thymic and marrow stromal cells | T and B lymphopoiesis |
| IL-8 | Monocytes, neutrophils, macrophages, | Chemoattractant (chemokine) for |
| | and NK cells. | neutrophils, basophils and T-cells; |
| | | activates neutrophils to degranulate. |
| IL-9 | T cells | hematopoietic and thymopoietic effects |
| IL-10 | activated TH₂ cells, CD8⁺ T and B | inhibits cytokine production, promotes B |
| | cells, macrophages | cell proliferation and antibody production, |
| | | suppresses cellular immunity, mast cell |
| | | growth |
| IL-11 | stromal cells | synergisitc hematopoietic and |
| | | thrombopoietic effects |
| IL-12 | B cells, macrophages | proliferation of NK cells, INF-g |
| | | production, promotes cell-mediated |
| | | immune functions |
| IL-13 | TH₂ cells | IL-4-like activities |
| TumorNecrosis | Primarily activated macrophages. | Once called cachectin; induces the |
| Factor | | expression of other autocrine growth |
| TNF-α | | factors, increases cellular responsiveness |
| | | to growth factors; induces signaling |
| | | pathways that lead to proliferation; |
| | | induces expression of a number of nuclear |
| | | proto-oncogenes as well as of several |
| | | interleukins. |
| (TNF-β) | T-lymphocytes, particularly cytotoxic | Also called lymphotoxin; kills a number |
| | T-lymphocytes (CTL cells); induced | of different cell types, induces terminal |
| | by IL-2 and antigen-T-Cell receptor | differentiation in others; inhibits |
| | interactions. | lipoprotein lipase present on the surface |
| | | of vascular endothelial cells. |
| Interferons | macrophages, neutrophils and some | Known as type I interferons; antiviral |
| INF-a and -b | somatic cells | effect; induction of class I MHC on all |
| | | somatic cells; activation of NK cells and |
| | | macrophages. |
| Interferon | Primarily CD8+ T-cells, activated TH₁ | Type II interferon; induces of class I |
| INF-γ | and NK cells | MHC on all somatic cells, induces class II |
| | | MHC on APCs and somatic cells, |
| | | activates macrophages, neutrophils, NK |
| | | cells, promotes cell-mediated immunity, |
| | | enhances ability of cells to present |
| | | antigens to T-cells; antiviral effects. |
| Colony | | Stimulate the proliferation of specific |
| Stimulating | | pluripotent stem cells of the bone marrow |
| Factors (CSFs) | | in adults. |
| Granulocyte- | | Specific for proliferative effects on cells |
| CSF (G-CSF) | | of the granulocyte lineage; proliferative |
| | | effects on both classes of lymphoid cells. |
| Macrophage- | | Specific for cells of the macrophage |
| CSF (M-CSF) | | lineage. |
| Granulocyte- | | Proliferative effects on cells of both the |
| MacrophageCSF | | macrophage and granulocyte lineages. |
| (GM-CSF) | | |

Other cytokines of interest that may be produced by the cells, cell lines, and cell cultures of the present invention described herein include adhesion molecules (R & D Systems, Adhesion Molecules I (1996), at http://www.rndsystems.com); angiogenin (U.S. Pat. No. 4,721,672; Moener et al., 226 Eur. J. Biochem. 483 (1994)); annexin V (Cookson et al., 20 Genomics 463 (1994); Grundmann et al., 85 PNAS 3708 (1988); U.S. Pat. No. 5,767,247); caspases (U.S. Pat. No. 6,214,858; Thornberry et al., 281 Science 1312 (1998)); chemokines (U.S. Pat. Nos. 6,174,995; 6,143,289; Sallusto et al., 18 Annu. Rev. Immunol. 593 (2000); Kunkel et al., 59 J. Leukocyte Biol. 81 (1996)); endothelin (U.S. Pat. Nos. 6,242,485; 5,294,569; 5,231,166); eotaxin (U.S. Pat. No. 6,271,347; Ponath et al., 97(3) J. Clin. Invest. 604-612 (1996)); Flt-3 (U.S. Pat. No. 6,190,655); heregulins (U.S. Pat. Nos. 6,284,535; 6,143,740; 6,136,558; 5,859,206; 5,840,525); Leptin (Leroy et al., 271(5) J. Biol. Chem. 2365 (1996); Maffei et al., 92 PNAS 6957 (1995); Zhang Y. et al. 372 Nature 425-32 (1994)); Macrophage Stimulating Protein (MSP) (U.S. Pat. Nos. 6,248,560; 6,030,949; 5,315,000); Pleiotrophin/Midkine (PTN/MK) (Pedraza et al., 117 J. Biochem. 845 (1995); Tamura et al., 3 Endocrine 21 (1995); U.S. Pat. No. 5,210,026; Kadomatsu et al., 151 Biochem. Biophys. Res. Commun. 1312 (1988)); STAT proteins (U.S. Pat. Nos. 6,030,808; 6,030,780; Darnell et al., 277 Science 1630-1635 (1997)); Tumor Necrosis Factor Family (Cosman, 7 Blood Cell Biochem. (Whetten et al., eds., Plenum Press, New York, 1996); Gruss et al., 85 Blood 3378 (1995); Beutler et al., 7 Annu. Rev. Immunol. 625 (1989); Aggarwal et al., 260 J. Biol. Chem. 2345 (1985); Pennica et al., 312 Nature 724 (1984)).

The present invention may also be used to produce recombinant forms of blood proteins, a generic name for a vast group of proteins generally circulating in blood plasma, and important for regulating coagulation and clot dissolution. See, e.g., Haematologic Technologies, Inc., HTI Catalog, at www.haemtech.com. Table 4 introduces, in a non-limiting fashion, some of the blood proteins contemplated by the present invention:

TABLE 4
Blood Proteins

| Protein | Principle Activity | Reference |
|---|---|---|
| Factor V | In coagulation, this glycoprotein pro- | Mann et al., 57 ANN. REV. BIOCHEM. |
| | cofactor, is converted to active cofactor, | 915 (1988); see also Nesheim et al., 254 |
| | factor Va, via the serine protease α- | J. BIOL. CHEM. 508 (1979); Tracy et al., |
| | thrombin, and less efficiently by its | 60 BLOOD 59 (1982); Nesheim et al., 80 |
| | serine protease cofactor Xa. The | METHODS ENZYMOL. 249 (1981); Jenny |
| | prothrombinase complex rapidly | et al., 84 PNAS 4846 (1987). |
| | converts zymogen prothrombin to the | |
| | active serine protease, α-thrombin. | |
| | Down regulation of prothrombinase | |
| | complex occurs via inactivation of Va | |
| | by activated protein C. | |
| Factor VII | Single chain glycoprotein zymogen. | See generally, Broze et al., 80 METHODS |
| | Proteolytic activation yields enzyme | ENZYMOL. 228 (1981); Bajaj et al., 256 |
| | factor VIIa, which binds to integral | J. BIOL. CHEM. 253 (1981); Williams et |
| | membrane protein tissue factor, forming | al., 264 J. BIOL. CHEM. 7536 (1989); |
| | an enzyme complex that converts factor | Kisiel et al., 22 THROMBOSIS RES. 375 |
| | X to Xa. Also known as extrinsic factor | (1981); Seligsohn et al., 64 J. CLIN. |
| | Xase complex. Conversion of VII to | INVEST. 1056 (1979); Lawson et al., 268 |
| | VIIa catalyzed by a number of proteases | J. BIOL. CHEM. 767 (1993). |
| | including thrombin, factors IXa, Xa, | |
| | XIa, and XIIa. Rapid activation also | |
| | occurs when VII combines with tissue | |
| | factor in the presence of Ca, likely | |
| | initiated by a small amount of pre- | |
| | existing VIIa. Not readily inhibited by | |
| | antithrombin III/heparin alone, but is | |
| | inhibited when tissue factor added. | |
| Factor IX | Zymogen factor IX, a single chain | Thompson, 67 BLOOD 565 (1986); |
| | vitamin K-dependent glycoprotein, | HEDNER ET AL., HEMOSTASIS AND |
| | made in liver. Binds to negatively | THROMBOSIS 39-47 (Colman et al., eds., |
| | charged phospholipid surfaces. | 2^{nd} ed. J. P. Lippincott Co., Philadelphia, |
| | Activated by factor XIα or the factor | 1987); Fujikawa et al., 45 METHODS IN |
| | VIIa/tissue factor/phospholipid | ENZYMOLOGY 74 (1974). |
| | complex. Cleavage at one site yields the | |
| | intermediate IXα, subsequently | |
| | converted to fully active form IXaβ by | |
| | cleavage at another site. Factor IXaβ is | |
| | the catalytic component of the “intrinsic | |
| | factor Xase complex” (factor | |
| | VIIIa/IXa/Ca²⁺/phospholipid) that | |
| | proteolytically activates factor X to | |
| | factor Xa. | |
| Factor X | Vitamin K-dependent protein zymogen, | See Davie et al., 48 ADV. ENZYMOL 277 |
| | made in liver, circulates in plasma as a | (1979); Jackson, 49 ANN. REV. |
| | two chain molecule linked by a disulfide | BIOCHEM. 765 (1980); see also |
| | bond. Factor Xa (activated X) serves as | Fujikawa et al., 11 BIOCHEM. 4882 |
| | the enzyme component of | (1972); Discipio et al., 16 BIOCHEM. |
| | prothrombinase complex, responsible | 698 (1977); Discipio et al., 18 |
| | for rapid conversion of prothrombin to | BIOCHEM. 899 (1979); Jackson et al., 7 |
| | thrombin. | BIOCHEM. 4506 (1968); McMullen et |
| | | al., 22 BIOCHEM. 2875 (1983). |
| Factor XI | Liver-made glycoprotein homodimer | Thompson et al., 60 J. CLIN. INVEST. |
| | circulates, in a non-covalent complex | 1376 (1977); Kurachi et al., 16 |
| | with high molecular weight kininogen, | BIOCHEM. 5831 (1977); Bouma et al., |
| | as a zymogen, requiring proteolytic | 252 J. BIOL. CHEM. 6432 (1977); |
| | activation to acquire serine protease | Wuepper, 31 FED. PROC. 624 (1972); |
| | activity. Conversion of factor XI to | Saito et al., 50 BLOOD 377 (1977); |
| | factor XIa is catalyzed by factor XIIa. | Fujikawa et al., 25 BIOCHEM. 2417 |
| | XIa unique among the serine proteases, | (1986); Kurachi et al., 19 BIOCHEM. |
| | since it contains two active sites per | 1330 (1980); Scott et al., 69 J. CLIN. |
| | molecule. Works in the intrinsic | INVEST. 844 (1982). |
| | coagulation pathway by catalyzing | |
| | conversion of factor IX to factor IXa. | |
| | Complex form, factor XIa/HMWK, | |
| | activates factor XII to factor XIIa and | |
| | prekallikrein to kallikrein. Major | |
| | inhibitor of XIa is a₁-antitrypsin and | |
| | to lesser extent, antithrombin-III. | |
| | Lack of factor XI procoagulant activity | |
| | causes bleeding disorder: plasma | |
| | thromboplastin antecedent deficiency. | |
| Factor XII | Glycoprotein zymogen. Reciprocal | SCHMAIER ET AL., HEMOSTASIS & |
| (Hageman | activation of XII to active serine | THROMBOSIS 18-38 (Colman et al., eds., |
| Factor) | protease factor XIIa by kallikrein is | J.B. Lippincott Co., Philadelphia, 1987); |
| | central to start of intrinsic coagulation | DAVIE, HEMOSTASIS & THROMBOSIS |
| | pathway. Surface bound α-XIIa activates | 242-267 (Colman et al., eds., J.B. |
| | factor XI to XIa. Secondary cleavage of | Lippincott Co., Philadelphia, 1987). |
| | α-XIIa by kallikrein yields β-XIIa, and | |
| | catalyzes solution phase activation of | |
| | kallikrein, factor VII and the classical | |
| | complement cascade. | |
| Factor XIII | Zymogenic form of glutaminyl-peptide | See MCDONAUGH, HEMOSTASIS & |
| | γ-glutamyl transferase factor XIIIa | THROMBOSIS 340-357 (Colman et al., |
| | (fibrinoligase, plasma transglutaminase, | eds., J.B. Lippincott Co., Philadelphia, |
| | fibrin stabilizing factor). Made in the | 1987); Folk et al., 113 METHODS |
| | liver, found extracellularly in plasma | ENZYMOL. 364 (1985); Greenberg et al., |
| | and intracellularly in platelets, | 69 BLOOD 867 (1987). Other proteins |
| | megakaryocytes, monocytes, placenta, | known to be substrates for Factor XIIIa, |
| | uterus, liver and prostrate tissues. | that may be hemostatically important, |
| | Circulates as a tetramer of 2 pairs of | include fibronectin (Iwanaga et al., 312 |
| | nonidentical subunits (A₂B₂). Full | ANN. NY ACAD. SCI. 56 (1978)), a₂- |
| | expression of activity is achieved only | antiplasmin (Sakata et al., 65 J. CLIN. |
| | after the Ca²⁺ - and fibrin(ogen)- | INVEST. 290 (1980)), collagen (Mosher |
| | dependent dissociation of B subunit | et al., 64 J. CLIN. INVEST. 781 (1979)), |
| | dimer from A₂′ dimer. Last of the | factor V (Francis et al., 261 J. BIOL. |
| | zymogens to become activated in the | CHEM. 9787 (1986)), von Willebrand |
| | coagulation cascade, the only enzyme in | Factor (Mosher et al., 64 J. CLIN. |
| | this system that is not a serine protease. | INVEST. 781 (1979)) and |
| | XIIIa stabilizes the fibrin clot by | thrombospondin (Bale et al., 260 J. |
| | crosslinking the α and γ-chains of fibrin. | BIOL. CHEM. 7502 (1985); Bohn, 20 |
| | Serves in cell proliferation in wound | MOL. CELL BIOCHEM. 67 (1978)). |
| | healing, tissue remodeling, | |
| | atherosclerosis, and tumor growth. | |
| Fibrinogen | Plasma fibrinogen, a large glycoprotein, | FURLAN, FIBRINOGEN, IN HUMAN |
| | disulfide linked dimer made of 3 pairs of | PROTEIN DATA, (Haeberli, ed., VCH |
| | non-identical chains (Aa, Bb and g), | Publishers, N.Y., 1995); DOOLITTLE, in |
| | made in liver. Aa has N-terminal peptide | HAEMOSTASIS & THROMBOSIS, 491-513 |
| | (fibrinopeptide A (FPA), factor XIIIa | (3rd ed., Bloom et al., eds., Churchill |
| | crosslinking sites, and 2 phosphorylation | Livingstone, 1994); HANTGAN ET AL., in |
| | sites. Bb has fibrinopeptide B (FPB), 1 | HAEMOSTASIS & THROMBOSIS 269-89 |
| | of 3 N-linked carbohydrate moieties, | (2^{nd} ed., Forbes et al., eds., Churchill |
| | and an N-terminal pyroglutamic acid. | Livingstone, 1991). |
| | The g chain contains the other N-linked | |
| | glycos. site, and factor XIIIa cross- | |
| | linking sites. Two elongated subunits | |
| | ((AaBbg)₂) align in an antiparallel way | |
| | forming a trinodular arrangement of the | |
| | 6 chains. Nodes formed by disulfide | |
| | rings between the 3 parallel chains. | |
| | Central node (n-disulfide knot, E | |
| | domain) formed by N-termini of all 6 | |
| | chains held together by 11 disulfide | |
| | bonds, contains the 2 IIa-sensitive sites. | |
| | Release of FPA by cleavage generates | |
| | Fbn I, exposing a polymerization site on | |
| | Aa chain. These sites bind to regions on | |
| | the D domain of Fbn to form proto- | |
| | fibrils. Subsequent IIa cleavage of FPB | |
| | from the Bb chain exposes additional | |
| | polymerization sites, promoting lateral | |
| | growth of Fbn network. Each of the 2 | |
| | domains between the central node and | |
| | the C-terminal nodes (domains D and E) | |
| | has parallel a-helical regions of the Aa, | |
| | Bb and g chains having protease- | |
| | (plasmin-) sensitive sites. Another major | |
| | plasmin sensitive site is in hydrophilic | |
| | preturbance of a-chain from C-terminal | |
| | node. Controlled plasmin degradation | |
| | converts Fbg into fragments D and E. | |
| Fibronectin | High molecular weight, adhesive, | Skorstengaard et al., 161 EUR. J. |
| | glycoprotein found in plasma and | BIOCHEM. 441 (1986); Kornblihtt et al., |
| | extracellular matrix in slightly different | 4 EMBO J. 1755 (1985); Odermatt et |
| | forms. Two peptide chains | al., 82 PNAS 6571 (1985); Hynes, 1 |
| | interconnected by 2 disulfide bonds, has | ANN. REV. CELL BIOL. 67 (1985); |
| | 3 different types of repeating | Mosher 35 ANN. REV. MED. 561 (1984); |
| | homologous sequence units. Mediates | Rouslahti et al., 44 CELL 517 (1986); |
| | cell attachment by interacting with cell | Hynes 48 CELL 549 (1987); Mosher 250 |
| | surface receptors and extracellular | BIOL. CHEM. 6614 (1975). |
| | matrix components. Contains an Arg- | |
| | Gly-Asp-Ser (RGDS) cell attachment- | |
| | promoting sequence, recognized by | |
| | specific cell receptors, such as those on | |
| | platelets. Fibrin-fibronectin complexes | |
| | stabilized by factor XIIIa-catalyzed | |
| | covalent cross-linking of fibronectin to | |
| | the fibrin a chain. | |
| b₂- | Also called b₂I and Apolipoprotein H. | See, e.g., Lozier et al., 81 PNAS 2640- |
| Glycoprotein I | Highly glycosylated single chain protein | 44 (1984); Kato & Enjyoi 30 BIOCHEM. |
| | made in liver. Five repeating mutually | 11687-94 (1997); Wurm, 16 INT'L J. |
| | homologous domains consisting of | BIOCHEM. 511-15 (1984); Bendixen et |
| | approximately 60 amino acids disulfide | al., 31 BIOCHEM. 3611-17 (1992); |
| | bonded to form Short Consensus | Steinkasserer et al., 277 BIOCHEM. J. |
| | Repeats (SCR) or Sushi domains. | 387-91 (1991); Nimpf et al., 884 |
| | Associated with lipoproteins, binds | BIOCHEM. BIOPHYS. ACTA 142-49 |
| | anionic surfaces like anionic vesicles, | (1986); Kroll et.al. 434 BIOCHEM. |
| | platelets, DNA, mitochondria, and | BIOPHYS. Acta 490-501 (1986); Polz et |
| | heparin. Binding can inhibit contact | al., 11 INT'L J. BIOCHEM. 265-73 |
| | activation pathway in blood coagulation. | (1976); McNeil et al., 87 PNAS 4120-24 |
| | Binding to activated platelets inhibits | (1990); Galli et a;. I LANCET 1544-47 |
| | platelet associated prothrombinase and | (1990); Matsuuna et al., II LANCET 177-78 |
| | adenylate cyclase activities. Complexes | (1990); Pengo et al., 73 THROMBOSIS |
| | between b₂I and cardiolipin have been | & HAEMOSTASIS 29-34 (1995). |
| | implicated in the anti-phospholipid | |
| | related immune disorders LAC and SLE. | |
| Osteonectin | Acidic, noncollagenous glycoprotein | Villarreal et al., 28 BIOCHEM. 6483 |
| | (Mr = 29,000) originally isolated from | (1989); Tracy et al., 29 INT'L J. |
| | fetal and adult bovine bone matrix. May | BIOCHEM. 653 (1988); Romberg et al., |
| | regulate bone metabolism by binding | 25 BIOCHEM. 1176 (1986); Sage & |
| | hydroxyapatite to collagen. Identical to | Bornstein 266 J. BIOL. CHEM. 14831 |
| | human placental SPARC. An alpha | (1991); Kelm & Mann 4 J. BONE MIN. |
| | granule component of human platelets | RES. 5245 (1989); Kelm et al., 80 |
| | secreted during activation. A small | BLOOD 3112 (1992). |
| | portion of secreted osteonectin | |
| | expressed on the platelet cell surface in | |
| | an activation-dependent manner | |
| Plasminogen | Single chain glycoprotein zymogen with | See Robbins, 45 METHODS IN |
| | 24 disulfide bridges, no free sulfhydryls, | ENZYMOLOGY 257 (1976); COLLEN, |
| | and 5 regions of internal sequence | 243-258 BLOOD COAG. (Zwaal et al., |
| | homology, “kringles”, each five triple- | eds., Elsevier, New York, 1986); see |
| | looped, three disulfide bridged, and | also Castellino et al., 80 METHODS IN |
| | homologous to kringle domains in t-PA, | ENZYMOLOGY 365 (1981); Wohl et al., |
| | u-PA and prothrombin. Interaction of | 27 THROMB. RES. 523 (1982); Barlow et |
| | plasminogen with fibrin and α2- | al., 23 BIOCHEM. 2384 (1984); |
| | antiplasmin is mediated by lysine | SOTTRUP-JENSEN ET AL., 3 PROGRESS IN |
| | binding sites. Conversion of | CHEM. FIBRINOLYSIS & THROMBOLYSIS |
| | plasminogen to plasmin occurs by | 197-228 (Davidson et al., eds., Raven |
| | variety of mechanisms, including | Press, New York, 1975). |
| | urinary type and tissue type | |
| | plasminogen activators, streptokinase, | |
| | staphylokinase, kallikrein, factors IXa | |
| | and XIIa, but all result in hydrolysis at | |
| | Arg560-Val561, yielding two chains | |
| | that remain covalently associated by a | |
| | disulfide bond. | |
| tissue | t-PA, a serine endopeptidase synthesized | See Plasminogen. |
| Plasminogen | by endothelial cells, is the major | |
| Activator | physiologic activator of plasminogen in | |
| | clots, catalyzing conversion of | |
| | plasminogen to plasmin by hydrolising a | |
| | specific arginine-alanine bond. Requires | |
| | fibrin for this activity, unlike the kidney- | |
| | produced version, urokinase-PA. | |
| Plasmin | See Plasminogen. Plasmin, a serine | See Plasminogen. |
| | protease, cleaves fibrin, and activates | |
| | and/or degrades compounds of | |
| | coagulation, kinin generation, and | |
| | complement systems. Inhibited by a | |
| | number of plasma protease inhibitors in | |
| | vitro. Regulation of plasmin in vivo | |
| | occurs mainly through interaction with | |
| | a₂-antiplasmin, and to a lesser extent, a₂- | |
| | macroglobulin. | |
| Platelet Factor-4 | Low molecular weight, heparin-binding | Rucinski et al., 53 BLOOD 47 (1979); |
| | protein secreted from agonist-activated | Kaplan et al., 53 BLOOD 604 (1979); |
| | platelets as a homotetramer in complex | George 76 BLOOD 859 (1990); Busch et |
| | with a high molecular weight, | al., 19 THROMB. RES. 129 (1980); Rao |
| | proteoglycan, carrier protein. Lysine- | et al., 61 BLOOD 1208 (1983); Brindley, |
| | rich, COOH-terminal region interacts | et al., 72 J. CLIN. INVEST. 1218 (1983); |
| | with cell surface expressed heparin-like | Deuel et al., 74 PNAS 2256 (1981); |
| | glycosaminoglycans on endothelial | Osterman et al., 107 BIOCHEM. |
| | cells, PF-4 neutralizes anticoagulant | BIOPHYS. RES. COMMUN. 130 (1982); |
| | activity of heparin exerts procoagulant | Capitanio et al., 839 BIOCHEM. |
| | effect, and stimulates release of | BIOPHYS. ACTA. 161 (1985). |
| | histamine from basophils. Chemotactic | |
| | activity toward neutrophils and | |
| | monocytes. Binding sites on the platelet | |
| | surface have been identified and may be | |
| | important for platelet aggregation. | |
| Protein C | Vitamin K-dependent zymogen, protein | See Esmon, 10 PROGRESS IN THROMB. |
| | C, made in liver as a single chain | & HEMOSTS. 25 (1984); Stenflo, 10 |
| | polypeptide then converted to a disulfide | SEMIN. IN THROMB. & HEMOSTAS. 109 |
| | linked heterodimer. Cleaving the heavy | (1984); Griffen et al., 60 BLOOD 261 |
| | chain of human protein C converts the | (1982); Kisiel et al., 80 METHODS |
| | zymogen into the serine protease, | ENZYMOL. 320 (1981); Discipio et al., |
| | activated protein C. Cleavage catalyzed | 18 BIOCHEM. 899 (1979). |
| | by a complex of α-thrombin and | |
| | thrombomodulin. Unlike other vitamin | |
| | K dependent coagulation factors, | |
| | activated protein C is an anticoagulant | |
| | that catalyzes the proteolytic | |
| | inactivation of factors Va and VIIIa, and | |
| | contributes to the fibrinolytic response | |
| | by complex formation with plasminogen | |
| | activator inhibitors. | |
| Protein S | Single chain vitamin K-dependent | Walker, 10 SEMIN. THROMB. |
| | protein functions in coagulation and | HEMOSTAS. 131 (1984); Dahlback et al., |
| | complement cascades. Does not | 10 SEMIN. THROMB. HEMOSTAS. 139 |
| | possess the catalytic triad. Complexes | (1984); Walker, 261 J. BIOL. CHEM. |
| | to C4b binding protein (C4BP) and to | 10941 (1986). |
| | negatively charged phospholipids, | |
| | concentrating C4BP at cell surfaces | |
| | following injury. Unbound S serves as | |
| | anticoagulant cofactor protein with | |
| | activated Protein C. A single cleavage | |
| | by thrombin abolishes protein S cofactor | |
| | activity by removing gla domain. | |
| Protein Z | Vitamin K-dependent, single-chain | Sejima et al., 171 BIOCHEM. |
| | protein made in the liver. Direct | BIOPHYSICS RES. COMM. 661 (1990); |
| | requirement for the binding of thrombin | Hogg et al., 266 J. BIOL. CHEM. 10953 |
| | to endothelial phospholipids. Domain | (1991); Hogg et al., 17 BIOCHEM. |
| | structure similar to that of other vitamin | BIOPHYSICS RES. COMM. 801 (1991); |
| | K-dependant zymogens like factors VII, | Han et al., 38 BIOCHEM. 11073 (1999); |
| | IX, X, and protein C. N-terminal region | Kemkes-Matthes et al., 79 THROMB. |
| | contains carboxyglutamic acid domain | RES. 49 (1995). |
| | enabling phospholipid membrane | |
| | binding. C-terminal region lacks | |
| | “typical” serine protease activation site. | |
| | Cofactor for inhibition of coagulation | |
| | factor Xa by serpin called protein Z- | |
| | dependant protease inhibitor. Patients | |
| | diagnosed with protein Z deficiency | |
| | have abnormal bleeding diathesis during | |
| | and after surgical events. | |
| Prothrombin | Vitamin K-dependent, single-chain | Mann et al., 45 METHODS IN |
| | protein made in the liver. Binds to | ENZYMOLOGY 156 (1976); |
| | negatively charged phospholipid | MAGNUSSON ET AL., PROTEASES IN |
| | membranes. Contains two “kringle” | BIOLOGICAL CONTROL 123-149 (Reich |
| | structures. Mature protein circulates in | et al., eds. Cold Spring Harbor Labs., |
| | plasma as a zymogen and, during | New York, 1975); Discipio et al., 18 |
| | coagulation, is proteolytically activated | BIOCHEM. 899 (1979). |
| | to the potent serine protease α-thrombin. | |
| α-Thrombin | See Prothrombin. During coagulation, | 45 METHODS ENZYMOL. 156 (1976). |
| | thrombin cleaves fibrinogen to form | |
| | fibrin, the terminal proteolytic step in | |
| | coagulation, forming the fibrin clot. | |
| | Thrombin also responsible for feedback | |
| | activation of procofactors V and VIII. | |
| | Activates factor XIII and platelets, | |
| | functions as vasoconstrictor protein. | |
| | Procoagulant activity arrested by | |
| | heparin cofactor II or the antithrombin | |
| | III/heparin complex, or complex | |
| | formation with thrombomodulin. | |
| | Formation of thrombin/thrombomodulin | |
| | complex results in inability of thrombin | |
| | to cleave fibrinogen and activate factors | |
| | V and VIII, but increases the efficiency | |
| | of thrombin for activation of the | |
| | anticoagulant, protein C. | |
| b-Thromboglobulin | Low molecular weight, heparin-binding, | See, e.g., George 76 BLOOD 859 (1990); |
| | platelet-derived tetramer protein, | Holt & Niewiarowski 632 BIOCHIM. |
| | consisting of four identical peptide | BIOPHYS. ACTA. 284 (1980); |
| | chains. Lower affinity for heparin than | Niewiarowski et al., 55 BLOOD 453 |
| | PF-4. Chemotactic activity for human | (1980); Varma et al., 701 BIOCHIM. |
| | fibroblasts, other functions unknown. | BIOPHYS. ACTA. 7 (1982); Senior et al., |
| | | 96 J. CELL. BIOL. 382 (1983). |
| Thrombopoietin | Human TPO (Thrombopoietin, Mpl- | Horikawa et al., 90(10) BLOOD 4031-38 |
| | ligand, MGDF) stimulates the | (1997); de Sauvage et al., 369 NATURE |
| | proliferation and maturation of | 533-58 (1995). |
| | megakaryocytes and promotes increased | |
| | circulating levels of platelets in vivo. | |
| | Binds to c-Mpl receptor. | |
| Thrombospondin | High-molecular weight, heparin-binding | Dawes et al., 29 THROMB. RES. 569 |
| | glycoprotein constituent of platelets, | (1983); Switalska et al., 106 J. LAB. |
| | consisting of three, identical, disulfide- | CLIN. MED. 690 (1985); Lawler et al. |
| | linked polypeptide chains. Binds to | 260 J. BIOL. CHEM. 3762 (1985); Wolff |
| | surface of resting and activated platelets, | et al., 261 J. BIOL. CHEM. 6840 (1986); |
| | may effect platelet adherence and | Asch et al., 79 J. CLIN. CHEM. 1054 |
| | aggregation. An integral component of | (1987); Jaffe et al., 295 NATURE 246 |
| | basement membrane in different tissues. | (1982); Wright et al., 33 J. HISTOCHEM. |
| | Interacts with a variety of extracellular | CYTOCHEM. 295 (1985); Dixit et al., |
| | macromolecules including heparin, | 259 J. BIOL. CHEM. 10100 (1984); |
| | collagen, fibrinogen and fibronectin, | Mumby et al., 98 J. CELL. BIOL. 646 |
| | plasminogen, plasminogen activator, | (1984); Lahav et al, 145 EUR. J. |
| | and osteonectin. May modulate cell- | BIOCHEM. 151 (1984); Silverstein et al, |
| | matrix interactions. | 260 J. BIOL. CHEM. 10346 (1985); |
| | | Clezardin et al. 175 EUR. J. BIOCHEM. |
| | | 275 (1988). |
| Von Willebrand | Multimeric plasma glycoprotein made of | Hoyer, 58 BLOOD 1 (1981); Ruggeri & |
| Factor | identical subunits held together by | Zimmerman 65 J. CLIN. INVEST. 1318 |
| | disulfide bonds. During normal | (1980); Hoyer & Shainoff, 55 BLOOD |
| | hemostasis, larger multimers of vWF | 1056 (1980); Meyer et al., 95 J. LAB. |
| | cause platelet plug formation by forming | CLIN. INVEST. 590 (1980); Santoro, 21 |
| | a bridge between platelet glycoprotein | THROMB. RES. 689 (1981); Santoro & |
| | IB and exposed collagen in the | Cowan, 2 COLLAGEN RELAT. RES. 31 |
| | subendothelium. Also binds and | (1982); Morton et al., 32 THROMB. RES. |
| | transports factor VIII (antihemophilic | 545 (1983); Tuddenham et al., 52 BRIT. |
| | factor) in plasma. | J. HAEMATOL. 259 (1982). |

Additional blood proteins contemplated herein include the following human serum proteins, which may also be placed in another category of protein (such as hormone or antigen): Actin, Actinin, Amyloid Serum P, Apolipoprotein E, B2-Microglobulin, C-Reactive Protein (CRP), Cholesterylester transfer protein (CETP), Complement C3B, Ceruplasmin, Creatine Kinase, Cystatin, Cytokeratin 8, Cytokeratin 14, Cytokeratin 18, Cytokeratin 19, Cytokeratin 20, Desmin, Desmocollin 3, FAS (CD95), Fatty Acid Binding Protein, Ferritin, Filamin, Glial Filament Acidic Protein, Glycogen Phosphorylase Isoenzyme BB (GPBB), Haptoglobulin, Human Myoglobin, Myelin Basic Protein, Neurofilament, Placental Lactogen, Human SHBG, Human Thyroid Peroxidase, Receptor Associated Protein, Human Cardiac Troponin C, Human Cardiac Troponin I, Human Cardiac Troponin T, Human Skeletal Troponin I, Human Skeletal Troponin T, Vimentin, Vinculin, Transferrin Receptor, Prealbumin, Albumin, Alpha-1-Acid Glycoprotein, Alpha-1-Antichymotrypsin, Alpha-1-Antitrypsin, Alpha-Fetoprotein, Alpha-1-Microglobulin, Beta-2-microglobulin, C-Reactive Protein, Haptoglobulin, Myoglobulin, Prealbumin, PSA, Prostatic Acid Phosphatase, Retinol Binding Protein, Thyroglobulin, Thyroid Microsomal Antigen, Thyroxine Binding Globulin, Transferrin , Troponin I, Troponin T, Prostatic Acid Phosphatase, Retinol Binding Globulin (RBP). All of these proteins, and sources thereof, are known in the art.

The cells, cell lines, and cell cultures of the present invention may also be used for the production of neurotransmitters, or functional portions thereof. Neurotransmitters are compounds made by neurons and used by them to transmit signals to the other neurons or non-neuronal cells (e.g., skeletal muscle, myocardium, pineal glandular cells) that they innervate. Neurotransmitters produce their effects by being released into synapses when their neuron of origin fires (i.e., becomes depolarized) and then attaching to receptors in the membrane of the post-synaptic cells. This causes changes in the fluxes of particular ions across that membrane, making cells more likely to become depolarized, if the neurotransmitter happens to be excitatory, or less likely if it is inhibitory. Neurotransmitters can also produce their effects by modulating the production of other signal-transducing molecules (“second messengers”) in the post-synaptic cells. See generally Cooper, Bloom & Roth, The Biochem. Basis of Neuropharmacology (7th Ed. Oxford Univ. Press, NYC, 1996); http://web.indstate.edu/thcme/mwking/nerves. Neurotransmitters contemplated in the present invention include, but are not limited to, endorphins (such as leu-enkephalin, morphiceptin, substance P), corticotropin releasing hormone, adrenocorticotropic hormone, vasopressin, giractide, peptide neurotransmitters derived from pre-opiomelanocortin, and N-acetylaspartylglutamate, the most prevalent and widely distributed peptide neurotransmitter in the mammalian nervous system. See Neale et al. 75 J. Neurochem. 443-52 (2000).

Numerous other proteins or peptides may be produced by the cells, cell lines, and cell cultures of the present invention described herein. Table 5 presents a non-limiting list and description of some pharmacologically active peptides which may be produced by such cells.

TABLE 5
Pharmacologically active peptides

| Binding partner/ | | |
|---|---|---|
| Protein of interest | | |
| (form of peptide) | Pharmacological activity | Reference |
| EPO receptor | EPO mimetic | Wrighton et al., 273 SCIENCE 458-63 |
| (intrapeptide | | (1996); U.S. Pat. No. 5,773,569. |
| disulfide-bonded) | | |
| EPO receptor | EPO mimetic | Livnah et al., 273 SCIENCE 464-71 |
| (C-terminally cross- | | (1996); Wrighton et al., 15 NATURE |
| linked dimer) | | BIOTECHNOLOGY 1261-5 (1997); WO |
| | | 96/40772. |
| EPO receptor | EPO mimetic | Naranda et al., 96 PNAS 7569-74 (1999). |
| (linear) | | |
| c-Mpl | TPO-mimetic | Cwirla et al., 276 SCIENCE 1696-9 (1997); |
| (linear) | | U.S. Pat. Nos. 5,932,946; 5,869,451. |
| c-Mpl | TPO-mimetic | Cwirla et al., 276 SCIENCE 1696-9 (1997). |
| (C-terminally cross- | | |
| linked dimer) | | |
| (disulfide-linked | stimulation of | Paukovits et al., 364 HOPPE-SEYLERS Z. |
| dimer) | hematopoesis | PHYSIOL. CHEM. 30311 (1984); |
| | (“G-CSF-mimetic”) | Laerurngal., 16 EXP. HEMAT. 274-80 |
| | | (1988). |
| (alkylene-linked dimer) | G-CSF-mimetic | Batnagar et al., 39 J. MED. CHEM. 38149 |
| | | (1996); Cuthbertson et al., 40 J. MED. |
| | | CHEM. 2876-82 (1997); King et al., 19 |
| | | EXP. HEMATOL. 481 (1991); King et al., |
| | | 86(Suppl. 1) BLOOD 309 (1995). |
| IL-1 receptor | inflammatory and | U.S. Pat. Nos. 5,880,096; 5,786,331; |
| (linear) | autoimmune diseases (“IL-1 | 5,608,035; Yanofsky et al., 93 PNAS |
| | antagonist” or “IL-1 ra- | 7381-6 (1996); Akeson et al., 271 J. BIOL. |
| | mimetic”) | CHEM. 30517-23 (1996); Wiekzorek et al., |
| | | 49 POL. J. PHARMACOL. 107-17 (1997); |
| | | Yanofsky, 93 PNAS 7381-7386 (1996). |
| Facteur thyrnique | stimulation of lymphocytes | Inagaki-Ohara et al., 171 CELLULAR |
| (linear) | (FTS-mimetic) | IMMUNOL. 30-40 (1996); Yoshida, 6 J. |
| | | IMMUNOPHARMACOL 141-6 (1984). |
| CTLA4 MAb | CTLA4-mimetic | Fukumoto et al., 16 NATURE BIOTECH. |
| (intrapeptide di-sulfide | | 267-70 (1998). |
| bonded) | | |
| TNF-a receptor | TNF-a antagonist | Takasaki et al., 15 NATURE BIOTECH. |
| (exo-cyclic) | | 1266-70 (1997); WO 98/53842. |
| TNF-a receptor | TNF-a antagonist | Chirinos-Rojas, 161(10) J. IMM., 5621-26 |
| (linear) | | (1998). |
| C3b | inhibition of complement | Sahu et al., 157 IMMUNOL. 884-91 (1996); |
| (intrapeptide di-sulfide | activation; autoimmune | Morikis et al., 7 PROTEIN SCI. 619-27 |
| bonded) | diseases (C3b antagonist) | (1998). |
| vinculin | cell adhesion processes, cell | Adey et al., 324 BIOCHEM. J. 523-8 |
| (linear) | growth, differentiation | (1997). |
| | wound healing, tumor | |
| | metastasis (“vinculin | |
| | binding”) | |
| C4 binding protein (C413P) | anti-thrombotic | Linse et al. 272 BIOL. CHEM. 14658-65 |
| (linear) | | (1997). |
| urokinase receptor | processes associated with | Goodson et al., 91 PNAS 7129-33 (1994); |
| (linear) | urokinase interaction with its | WO 97/35969. |
| | receptor (e.g. angiogenesis, | |
| | tumor cell invasion and | |
| | metastasis; (URK antagonist) | |
| Mdm2, Hdm2 | Inhibition of inactivation of | Picksley et al., 9 ONCOGENE 2523-9 |
| (linear) | p53 mediated by Mdm2 or | (1994); Bottger et al. 269 J. MOL. BIOL. |
| | hdm2; anti-tumor | 744-56 (1997); Bottger et al., 13 |
| | (“Mdm/hdm antagonist”) | ONCOGENE 13: 2141-7 (1996). |
| p21^{WAF1} | anti-tumor by mimicking the | Ball et al., 7 CURR. BIOL. 71-80 (1997). |
| (linear) | activity of p21^{WAF1} | |
| farnesyl transferase | anti-cancer by preventing | Gibbs et al., 77 CELL 175-178 (1994). |
| (linear) | activation of ras oncogene | |
| Ras effector domain | anti-cancer by inhibiting | Moodie et at., 10 TRENDS GENEL 44-48 |
| (linear) | biological function of the ras | (1994); Rodriguez et al., 370 NATURE |
| | oncogene | 527-532 (1994). |
| SH2/SH3 domains | anti-cancer by inhibiting | Pawson et al, 3 CURR. BIOL. 434-432 |
| (linear) | tumor growth with activated | (1993); Yu et al., 76 CELL 933-945 |
| | tyrosine kinases | (1994). |
| p16^{INK4} | anti-cancer by mimicking | Fahraeus et al., 6 CURR. BIOL. 84-91 |
| (linear) | activity of p16; e.g., | (1996). |
| | inhibiting cyclin D-Cdk | |
| | complex (“p, 16-mimetic”) | |
| Src, Lyn | inhibition of Mast cell | Stauffer et al., 36 BIOCHEM. 9388-94 |
| (linear) | activation, IgE-related | (1997). |
| | conditions, type I | |
| | hypersensitivity (“Mast cell | |
| | antagonist”). | |
| Mast cell protease | treatment of inflammatory | WO 98/33812. |
| (linear) | disorders mediated by | |
| | release of tryptase-6 (“Mast | |
| | cell protease inhibitors”) | |
| SH3 domains | treatment of SH3-mediated | Rickles et al., 13 EMBO J. 5598-5604 |
| (linear) | disease states (“SH3 | (1994); Sparks et al., 269 J. |
| | antagonist”) | BIOL. CHEM. 238536 (1994); |
| | | Sparks et al., 93 PNAS 1540-44 |
| | | (1996). |
| HBV core antigen (HBcAg) | treatment of HBV viral | Dyson & Muray, 92(6) PNAS |
| (linear) | antigen (HBcAg) infections | 2194-98 (1995). |
| | (“anti-HBV”) | |
| selectins | neutrophil adhesion | Martens et al., 270 J. BIOL. |
| (linear) | inflammatory diseases | CHEM. 21129-36 (1995); |
| | (“selectin antagonist”) | EP 0 714 912. |
| calmodulin | calmodulin | Pierce et al., 1 MOLEC. |
| (linear, cyclized) | antagonist | DIVEMILY 25965 (1995); |
| | | Dedman et al., 267 J. BIOL. |
| | | CHEM. 23025-30 (1993); Adey |
| | | & Kay, 169 GENE 133-34 |
| | | (1996). |
| integrins | tumor-homing; treatment for | WO 99/24462; WO 98/10795; |
| (linear, cyclized) | conditions related to | WO 97/08203; WO 95/14714; Kraft et al., |
| | integrin-mediated cellular | 274 J. BIOL. CHEM. 1979-85 (1999). |
| | events, including platelet | |
| | aggregation, thrombosis, | |
| | wound healing, osteoporosis, | |
| | tissue repair, angiogenesis | |
| | (e.g., for treatment of cancer) | |
| | and tumor invasion | |
| | (“integrin-binding”) | |
| fibronectin and extracellular | treatment of inflammatory | WO 98/09985. |
| matrix components of T-cells | and autoimmune conditions | |
| and macrophages | | |
| (cyclic, linear) | | |
| somatostatin and cortistatin | treatment or prevention of | EP 0 911 393. |
| (linear) | hormone-producing tumors, | |
| | acromegaly, giantism, | |
| | dementia, gastric ulcer, | |
| | tumor growth, inhibition of | |
| | hormone secretion, | |
| | modulation of sleep or | |
| | neural activity | |
| bacterial lipopoly-saccharide | antibiotic; septic shock; | U.S. Pat. No. 5,877,151. |
| (linear) | disorders modulatable by | |
| | CAP37 | |
| parclaxin, mellitin | antipathogenic | WO 97/31019. |
| (linear or cyclic) | | |
| VIP | impotence, neuro- | WO 97/40070. |
| (linear, cyclic) | degenerative disorders | |
| CTLs | cancer | EP 0 770 624. |
| (linear) | | |
| THF-gamma2 | | Burnstein, 27 BIOCHEM. 4066-71 (1988). |
| (linear) | | |
| Amylin | | Cooper, 84 PNAS 8628-32 (1987). |
| (linear) | | |
| Adreno-medullin | | Kitamura, 192 BBRC 553-60 (1993). |
| (linear) | | |
| VEGF | anti-angiogenic; cancer, | Fairbrother, 37 BIOCHEM. 17754-64 |
| (cyclic, linear) | rheumatoid arthritis, diabetic | (1998). |
| | retinopathy, psoriasis | |
| | (“VEGF antagonist”) | |
| MMP | inflammation and | Koivunen, 17 NATURE BIOTECH. 768-74 |
| (cyclic) | autoimmune disorders; | (1999). |
| | tumor growth (“MMP | |
| | inhibitor“) | |
| HGH fragment | | U.S. Pat. No. 5,869,452. |
| (linear) | | |
| Echistatin | inhibition of platelet | Gan, 263 J. BIOL. 19827-32 (1988). |
| | aggregation | |
| SLE autoantibody | SLE | WO 96/30057. |
| (linear) | | |
| GD1 alpha | suppression of tumor | Ishikawa et al., 1 FEBS LETT. 20-4 |
| | metastasis | (1998). |
| anti-phospholipid β-2 | endothelial cell activation, | Blank Mal., 96 PNAS 5164-8 (1999). |
| glycoprotein-1 (β2GPI) | anti-phospholipid syndrome | |
| antibodies | (APS), thromboembolic | |
| | phenomena, | |
| | thrombocytopenia, and | |
| | recurrent fetal loss | |
| T-Cell Receptor β chain | diabetes | WO 96/101214. |
| (linear) | | |

There are two pivotal cytokines in the pathogenesis of rheumatoid arthritis, IL-1 and TNF-α. They act synergistically to induce each other, other cytokines, and COX-2. Research suggests that IL-1 is a primary mediator of bone and cartilage destruction in rheumatoid arthritis patients, whereas TNF-α appears to be the primary mediator of inflammation.

In a preferred embodiment, arecombinant protein produced by the cells, cell lines, and cell cultures of the present invention binds to tumor necrosis factor alpha (TNF-α), a pro-inflamatory cytokine. U.S. Pat. Nos. 6,277,969; 6,090,382. Anti-TNF-α antibodies have shown great promise as therapeutics. For example, Infliximab, provided commercially as REMICADE® by Centocor, Inc. (Malvern, Pa.) has been used for the treatment of several chronic autoimmune diseases such as Crohn's disease and rheumatoid arthritis. See Centocor's pending U.S. patent applications, Ser. No. 09/920,137; Nos. 60/236,826; 60/223,369. See also Treacy, 19(4) Hum. Exp. Toxicol. 226-28 (2000); see also Chantry, 2(1) Curr. Opin. Anti-Inflammatory Immunomodulatory Invest. Drugs 31-34 (2000); Rankin et al., 34(4) Brit. J. Rheumatology 334-42 (1995). Preferably, any exposed amino acids of the TNFα-binding moiety of the protein produced by the cell culture of the present invention are those with minimal antigenicity in humans, such as human or humanized amino acid sequences. These peptide identities may be generated by screening libraries, as described above, by grafting human amino acid sequences onto murine-derived paratopes (Siegel et al., 7(1) Cytokine 15-25 (1995); WO 92/11383) or monkey-derived paratopes (WO 93/02108), or by utilizing xenomice (WO 96/34096). Alternatively, murine-derived anti-TNFα antibodies have exhibited efficacy. Saravolatz et al., 169(1) J. Infect. Dis. 214-17 (1994).

Alternatively, instead of being derived from an antibody, the TNFα binding moiety of the protein produced in the cells, cell lines, and cell cultures of the present invention may be derived from the TNFα receptor. For example, Etanercept is a recombinant, soluble TNFα receptor molecule that is administered subcutaneously and binds to TNFα in the patient's serum, rendering it biologically inactive. Etanercept is a dimeric fusion protein consisting of the extracellular ligand-binding portion of the human 75 kilodalton (p75) tumor necrosis factor receptor (TNFR) linked to the Fc portion of human IgG1. The Fc component of etanercept contains the C_{H}2 domain, the C_{H}3 domain and hinge region, but not the C_{H}1 domain of IgG1. Etanercept is produced by recombinant DNA technology in a Chinese hamster ovary (CHO) mammalian cell expression system. It consists of 934 amino acids and has an apparent molecular weight of approximately 150 kilodaltons. Etanercept may be obtained as ENBREL™, manufactured by Immunex Corp. (Seattle, Wash.). Etanercept may be efficacious in rheumatoid arthritis. Hughes et al., 15(6) BioDrugs 379-93 (2001).

Another form of human TNF receptor exists as well, identified as p55. Kalinkovich et al., J. Inferon & Cytokine Res. 15749-57 (1995). This receptor has also been explored for use in therapy. See, e.g., Qian et al. 118 Arch. Ophthalmol. 1666-71 (2000). A previous formulation of the soluble p55 TNF receptor had been coupled to polyethylene glycol [r-metHuTNFbp PEGylated dimer (TNFbp)], and demonstrated clinical efficacy but was not suitable for a chronic indication due to the development antibodies upon multiple dosing, which resulted in increased clearance of the drug. A second generation molecule was designed to remove the antigenic epitopes of TNFbp, and may be useful in treating patients with rheumatoid arthritis. Davis et al., Presented at Ann. European Cong. Rheumatology, Nice, France (Jun. 21-24, 2000).

IL-1 receptor antagonist (IL-1Ra) is a naturally occurring cytokine antagonist that demonstrates anti-inflammatory properties by balancing the destructive effects of IL-1α and IL-1β in rheumatoid arthritis but does not induce any intracellular response. Hence, in a preferred embodiment of the invention, the cell culture may produce IL-1Ra, or any structural or functional analog thereof. Two structural variants of IL-1Ra exist: a 17-kDa form that is secreted from monocytes, macrophages, neutrophils, and other cells (sIL-1Ra) and an 18-kDa form that remains in the cytoplasm of keratinocytes and other epithelial cells, monocytes, and fibroblasts (icIL-1Ra). An additional 16-kDa intracellular isoform of IL-1Ra exists in neutrophils, monocytes, and hepatic cells. Both of the major isoforms of IL-1Ra are transcribed from the same gene through the use of alternative first exons. The production of IL-1Ra is stimulated by many substances including adherent IgG, other cytokines, and bacterial or viral components. The tissue distribution of IL-1Ra in mice indicates that sIL-1Ra is found predominantly in peripheral blood cells, lungs, spleen, and liver, while icIL-1Ra is found in large amounts in skin. Studies in transgenic and knockout mice indicate that IL-1Ra is important in host defense against endotoxin-induced injury. IL-1Ra is produced by hepatic cells with the characteristics of an acute phase protein. Endogenous IL-1 Ra is produced in human autoimmumune and chronic inflammatory diseases. The use of neutralizing anti-IL-1Ra antibodies has demonstrated that endogenous IL-1Ra is an important natural antiinflammatory protein in arthritis, colitis, and granulomatous pulmonary disease. Patients with rheumatoid arthritis treated with IL-1Ra for six months exhibited improvements in clinical parameters and in radiographic evidence of joint damage. Arend et al., 16 Ann. Rev. Immunol. 27-55 (1998).

Yet another example of an IL-1Ra that may be produced by the cells, cell lines, and cell cultures described herein is a recombinant human version called interleukin-1 17.3 Kd met-IL1ra, or Anakinra, produced by Amgen, (San Francisco, Calif.) under the name KINERET™. Anakinra has also shown promise in clinical studies involving patients with rheumatoid arthritis. 65th Ann. Sci. Meeting of Am. College Rheumatology (Nov. 12, 2001).

In another embodiment of the invention, the protein produced by the cells, cell lines, and cell cultures of the present invention is interleukin 12 (IL-12) or an antagnoist thereof. IL-12 is a heterodimeric cytokine consisting of glycosylated polypeptide chains of 35 and 40 kD which are disulfide bonded. The cytokine is synthesized and secreted by antigen presenting cells, including dendritic cells, monocytes, macrophages, B cells, Langerhans cells and keratinocytes, as well as natural killer (NK) cells. IL-12 mediates a variety of biological processes and has been referred to as NK cell stimulatory factor (NKSF), T-cell stimulating factor, cytotoxic T-lymphocyte maturation factor and EBV-transformed B-cell line factor. Curfs et al., 10 Clin. Micro. Rev. 742-80 (1997). Interleukin-12 can bind to the IL-12 receptor expressed on the plasma membrane of cells (e.g., T cells, NK cell), thereby altering (e.g., initiating, preventing) biological processes. For example, the binding of IL-12 to the IL-12 receptor can stimulate the proliferation of pre-activated T cells and NK cells, enhance the cytolytic activity of cytotoxic T cells (CTL), NK cells and LAK (lymphokine activated killer) cells, induce production of gamma interferon (IFNγ) by T cells and NK cells and induce differentiation of naive Th0 cells into Th1 cells that produce IFNγ and IL-2. Trinchieri, 13 Ann. Rev. Immunology 251-76 (1995). In particular, IL-12 is vital for the generation of cytolytic cells (e.g., NK, CTL) and for mounting a cellular immune response (e.g., a Th1 cell mediated immune response). Thus, IL-12 is critically important in the generation and regulation of both protective immunity (e.g., eradication of infections) and pathological immune responses (e.g., autoimmunity). Hendrzak et al., 72 Lab. Investigation 619-37 (1995). Accordingly, an immune response (e.g., protective or pathogenic) can be enhanced, suppressed or prevented by manipulation of the biological activity of IL-12 in vivo, for example, by means of an antibody.

In another embodiment, the cells, cell lines, and cell cultures of the present invention produce an integrin. Integrins have been implicated in the angiogenic process, by which tumor cells form new blood vessels that provide tumors with nutrients and oxygen, carry away waste products, and to act as conduits for the metastasis of tumor cells to distant sites. Gastl et al., 54 Oncol. 177-84 (1997). Integrins are heterodimeric transmembrane proteins that play critical roles in cell adhesion to the extracellular matrix (ECM) which, in turn, mediates cell survival, proliferation and migration through intracellular signaling. The heterodimeric integrins are comprise of an alpha subunit and a beta subunit. Currently, there are 16 known alpha subunits, which include α1, α2 , α3, α4, α5, α6, α7, α8, α9, αD, αL, αM, αV, αX, αIIb, αIELb. There are 8 known beta subunits, which include β1, β2, β3, β4, β5, β6, β7, β8. Some of the integrin heterodimers include, but are not limited to, α1β1, α2β1, α3β1, α4β1, α5β1, α6β1, α7β1, α8β1, α9β1, α4β7, α6β4, αDβ2, αLβ2, αMβ2, αVβ1, αVβ3, αVβ5, αVβ6, αVβ8, αXβ2, αIIbβ3, αIELbβ7. See generally, Block et al., 13 Stem Cells 135-145 (1995); Schwartz et al., 1(1) Ann. Rev. Cell Dev. Biol. 549-599 (1995); Hynes, 69 Cell 11-25 (1992).

During angiogenesis, a number of integrins that are expressed on the surface of activated endothelial cells regulate critical adhesive interactions with a variety of ECM proteins to regulate distinct biological events such as cell migration, proliferation and differentiation. Specifically, the closely related but distinct integrins aVb3 and aVb5 have been shown to mediate independent pathways in the angiogenic process. An antibody generated against αVβ3 blocked basic fibroblast growth factor (bFGF) induced angiogenesis, whereas an antibody specific to αVβ5 inhibited vascular endothelial growth factor-induced (VEGF-induced) angiogenesis. Eliceiri et al., 103 J. Clin. Invest. 1227-30 (1999); Friedlander et al., 270 Science 1500-02 (1995).

In another preferred embodiment of the invention, the cells, cell lines, and cell cultures produce a glycoprotein IIb/IIIa receptor antagonist. More specifically, the final obligatory step in platelet aggregation is the binding of fibrinogen to an activated membrane-bound glycoprotein complex, GP IIb/IIIa. Platelet activators such as thrombin, collagen, epinephrine or ADP, are generated as an outgrowth of tissue damage. During activation, GP IIb/IIa undergoes changes in conformation that results in exposure of occult binding sites for fibrinogen. There are six putative recognition sites within fibrinogen for GP IIb/IlIa and thus fibrinogen can potentially act as a hexavalent ligand to crossing GP IIb/IIIa molecules on adjacent platelets. A deficiency in either fibrinogen or GP IIb/IIIa a prevents normal platelet aggregation regardless of the agonist used to activate the platelets. Since the binding of fibrinogen to its platelet receptor is an obligatory component of normal aggregation, GP IIb/IIIa is an attractive target for an antithrombotic agent.

Results from clinical trials of GP IIb/IIIa inhibitors support this hypothesis. The monoclonal antibody 7E3, which blocks the GP IIb/IIIa receptor, has been shown to be an effective therapy for the high risk angioplasty population. It is used as an adjunct to percutaneous transluminal coronary angioplasty or atherectomy for the prevention of acute cardiac ischemic complications in patients at high risk for abrupt closure of the treated coronary vessel. Although 7E3 blocks both the IIb/IIIa receptor and the αᵥβ₃ receptor, its ability to inhibit platelet aggregation has been attributed to its function as a IIb/IIIa receptor binding inhibitor. The IIb/IIIa receptor antagonist may be, but is not limited to, an antibody, a fragment of an antibody, a peptide, or an organic molecule. For example, the target-binding moiety may be derived from 7E3, an antibody with glycoprotein IIb/IIIa receptor antagonist activity. 7E3 is the parent antibody of c7E3, a F(ab′)₂ fragment known as abciximab, known commercially as REOPRO®, produced by Centocor, Inc (Malvern, Pa.). Abciximab binds and inhibits the adhesive receptors GPIIb/IIa and αᵥβ₃, leading to inhibition of platelet aggregation and thrombin generation, and the subsequent prevention of thrombus formation. U.S. Pat. Nos. 5,976,532; 5,877,006; 5,770,198; Coller, 78 Throm. Haemost. 730-35 (1997); Jordan et al., in New Therapeutic Agents in Thrombosis & Thrombolysis (Sasahara & Loscalzo, eds. Marcel Kekker, Inc. New York, 1997); Jordan et al., in Adhesion Receptors as Therapeutic Targets 281-305 (Horton, ed. CRC Press, New York, 1996).

Alternatively, the protein produced by the cells, cell lines, and cell cultures of the present invention may be a thrombolytic. For example, the thrombolytic may be tPA, or a functional variation thereof. RETAVASE®, produced by Centocor, Inc. (Malvern, Pa.), is a variant tPA with a prolonged half-life. Interestingly, in mice, the combination of Retavase and the IIb/IIIa receptor antagonist 7E3F(ab′)₂ markedly augmented the dissolution of pulmonary embolism. See U.S. Provisional Patent Application Serial No. 60/304409.

The cells, cell lines, and cell cultures of the present invention may also be used produce receptors, or fragments thereof, and activated receptors, i.e., recombinant peptides that mimic ligands associated with their corresponding receptors, or fragments thereof. These complexes may mimic activated receptors and thus affect a particular biological activity. Alternatively, the receptor can be genetically re-engineered to adopt the activated conformation. For example, the thrombin-bound conformation of fibrinopeptide A exhibits a strand-turn-strand motif, with a β-turn centered at residues Glu-11 and Gly-12. Molecular modeling analysis indicates that the published fibrinopeptide conformation cannot bind reasonably to thrombin, but that reorientation of two residues by alignment with bovine pancreatic trypsin inhibitor provides a good fit within the deep thrombin cleft and satisfies all of the experimental nuclear Overhauser effect data. Based on this analysis, a researchers were able to successfully design and synthesize hybrid peptide mimetic substrates and inhibitors that mimic the proposed β-turn structure. The results indicate that the turn conformation is an important aspect of thrombin specificity, and that the turn mimetic design successfully mimics the thrombin-bound conformation of fibrinopeptide. Nakanishi et al., 89(5) PNAS 1705-09 (1992).

Another example of activated-receptor moieties concerns the peptido mimetics of the erythropoietin (Epo) receptor. By way of background, the binding of Epo to the Epo receptor (EpoR) is crucial for production of mature red blood cells. The Epo-bound, activated EpoR is a dimer. See, e.g., Constantinescu et al., 98 PNAS 4379-84 (2001). In its natural state, the first EpoR in the dimer binds Epo with a high affinity whereas the second EpoR molecule binds to the complex with a low affinity. Bivalent anti-EpoR antibodies have been reported to activate EopR, probably by dimerization of the EpoR. Additionally, small synthetic peptides, that do not have any sequence homology with the Epo molecule, are also able to mimic the biologic effects of Epo but with a lower affinity. Their mechanism of action is probably also based on the capacity to produce dimerization of the EpoR. Hence, an embodiment of the present invention provides for a method of producing an activated EpoR mimetic using the disclosed cell culture system.

In another embodiment of the invention, the cells, cell lines, and cell cultures may be used to produce antimicrobial agents or portions thereof, which include antibacterial agents, antivirals agents, antifungal agents, antimycobacterial agents, and antiparasitic agents. Antibacterials include, but are not limited to, -lactam antibiotics (penicillin G, ampicillin, oxacillin), aminoglycosides (streptomycin, kanamycin,neomycin and gentamicin), and polypeptide antibiotics (colistin, polymyxin B). Antimycobacterial agents that may be produced by the present cell culture include streptomycin. Sanford et al., Guide to Antimicrobial Therapy (25th ed., Antimicrobial Therapy, Inc., Dallas, Tex., 1995).

In another embodiment of the invention, the cells, cell lines, and cell cultures may be used to produce a cell cycle protein or a functionally active portion of a cell cycle protein. These cell cycle proteins are known in the art, and include cyclins, such as G₁ cyclins, S-phase cyclins, M-phase cyclins, cyclin A, cyclin D and cyclin E; the cyclin-dependent kinases (CDKs), such as G₁ CDKs, S-phase CDKs and M-phase CDKs, CDK2, CDK4 and CDK 6; and the tumor suppressor genes such as Rb and p53. Cell cycle proteins also include those involved in apoptosis, such as Bcl-2 and caspase proteins; proteins associated with Cdc42 signaling, p70 S6 kinase and PAK regulation; and integrins, discussed elsewhere. Also included in the cell cycle proteins of the present invention are anaphase-promoting complex (APC) and other proteolytic enzymes. The APC triggers the events leading to destruction of the cohesins and thus allowing sister chromatids to separate, and degrades the mitotic (M-phase) cyclins. Cell cycle proteins also include p13, p27, p34, p60, p80, histone H1, centrosomal proteins, lamins, and CDK inhibitors. Other relevant cell cycle proteins include S-phase promoting factor, M-phase promoting factor that activates APC. Kimball, Kimball's Biology Pages, at http://www.ultranet.com/˜jkimball/BiologyPages.

The cells, cell lines, and cell cultures of the present invention may also produce a particular antigen or portion thereof. Antigens, in a broad sense, may include any molecule to which an antibody, or functional fragment thereof, binds. Such antigens may be pathogen derived, and be associated with either MHC class I or MHC class II reactions. These antigens may be proteinaceous or include carbohydrates, such as polysaccharides, glycoproteins, or lipids. Carbohydrate and lipid antigens are present on cell surfaces of all types of cells, including normal human blood cells and foreign, bacterial cell walls or viral membranes. See Sears, Immunology (W. H. Freeman & Co. and Sumanas, Inc., 1997), available on-line at http://www.whfreeman.com/immunology.

For example, recombinant antigens may be derived from a pathogen, such as a virus, bacterium, mycoplasm, fungus, parasite, or from another foreign substance, such as a toxin. Such bacterial antigens may include or be derived from Bacillus anthracis, Bacillus tetani, Bordetella pertusis; Brucella spp., Corynebacterium diphtheriae, Clostridium botulinum, Clostridium perfringens, Coxiella burnetii, Francisella tularensis, Mycobacterium leprae, Mycobacterium tuberculosis, Salmoniella typhimurium, Streptocceus pneumoniae, Escherichia coli, Haemophilus influenzae, Shigella spp., Staphylococcus aureus, Neisseria gonorrhoeae, Neisseria meningiditis, Treponema pallidum, Yersinia pestis, Vibrio cholerae. Often, the oligosaccharide structures of the outer cell walls of these microbes afford superior protective immunity, but must be conjugated to an appropriate carrier for that effect.

Viruses and viral antigens that are within the scope of the current invention include, but are not limited to, HBeAg, Hepatitis B Core, Hepatitis B Surface Antigen, Cytomegalovirus B, HIV-1 gag, HIV-1 nef, HIV-1 env, HIV-1 gp41-1, HIV-1 p24, HIV-1 MN gp120, HIV-2 env, HIV-2 gp 36, HCV Core, HCV NS4, HCV NS3, HCV p22 nucleocapsid, HPV L1 capsid, HSV-1 gD, HSV-1 gG, HSV-2 gG, HSV-II, Influenza A (H1N1), Influenza A (H3N2), Influenza B, Parainfluenza Virus Type 1, Epstein Barr virus capsid antigen, Epstein Barr virus, Poxviridae Variola major, Poxviridae Variola minor, Rotavirus, Rubella virus, Respiratory Syncytial Virus, Surface Antigens of the Syphilis spirochete, Mumps Virus Antigen, Varicella zoster Virus Antigen and Filoviridae.

Other parasitic pathogens such as Chlamydia trachomatis, Plasmodium falciparum, and Toxoplasma gondii may also provide the source for recombinant antigens produced by cells, cell lines, and cell cultures of the present invention.

Moreover, recombinant toxins, toxoids, or antigenic portions of either, may be produced by the cells, cell lines, and cell cultures presented herein. These include those recombinant forms of toxins produced natively by bacteria, such as diphteria toxin, tetanus toxin, botulin toxin and enterotoxin B and those produced natively by plants, such as Ricin toxin from the castor bean Ricinus cummunis. Other toxins and toxoids that may be generated recombinantly include those derived from other plants, snakes, fish, frogs, spiders, scorpions, blue-green algae, fungi, and snails.

Still other antigens that may be produced by the cells, cell lines, and cell cultures of the present invention may be those that serve as markers for particular cell types, or as targets for an agent interacting with that cell type. Examples include Human Leukocyte Antigens (HLA markers), MHC Class I and Class II, the numerous CD markers useful for identifying T-cells and the physiological states thereof. Alternatively, antigens may serve as “markers” for a particular disease or condition, or as targets of a therapeutic agent. Examples include, Prostate Specific Antigen, Pregnancy specific beta 1 glycoprotein (SP1), Carcinoembryonic Antigen (CEA), Thyroid Microsomal Antigen, and Urine Protein 1. Antigens may include those defined as “self” implicated in autoimmune diseases. Haptens, low molecular weight compounds such as peptides or antibiotics that are too small to cause an immune response unless they are coupled with much larger entities, may serve as antigens when coupled to a larger carrier molecule, and are thus within the scope of the present invention. See Roitt et al., Immunology (5th ed., 1998); Benjamini et al., Immunology, A Short Course (3rd ed., 1996).

The present invention further relates to business methods where the cells, cell lines, cell cultures and recombinant proteins derived therefrom are provided to customers. In a specific embodiment, a customer is provided with the cells, cell lines, or cell cultures of the present invention. In another embodiment, a customer is provided with the cells, cell lines, or cell cultures cell line of the present invention that are transfected with an expression vector encoding a recombinant protein. In yet another embodiment, a customer is provided with a recombinant protein purified from the cells, cell lines, or cell cultures cell line of the present invention.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

### Example 1

Transfection of Cell Line C463A with rTNV148B, a Human Antibody to Tumor Necrosis Factor Alpha (TNFα), to Create the C463A-Derived rTNV148B-production Cell Line Designated C524A.

The cell line C463A was further tested as a suitable host for the expression of recombinant proteins. This example describes the transfection and subsequent development of the C463A-derived rTNV148B production cell line designated C524A. rTNV148B is a totally human monoclonal antibody directed against TNFα, the genes for which were obtained using hybridoma techniques and transgenic mice.

Transfection and Screening

rTNV148B heavy chain expression vector, designated plasmid p1865, was linearized by digestion with Xho1 and rTNV148B light chain expression vector, designated plasmid p1860, was linearized using SaII restriction enzyme. Approximately 1×10⁷ C463A cells were transfected, with about 10 μg of the premixed linearized plasmids, by electroporation (200 V and 1180 uF). See Knight et al., 30 Molecular Immunolgy 1443 (1993). Following transfection, the cells were seeded at a viable cell density of 1×10⁴ cells/well in 96-well tissue culture dishes with IMDM, 15% FBS, 2 mM glutamine. After incubating the cells at 37° C., 5% CO₂ for about 40 hours, an equal volume of IMDM, 5% FBS, 2 mM glutamine and 2×MHX selection medium was added. The plates were incubated at 37° C., 5% CO₂ for about 2 weeks until colonies (primary transfectants) became visible.

Cell supernatants from wells in which there were visible colonies were assayed for human IgG by ELISA using a standard curve generated from protein-A column-purified rTNV148B human anti-TNF. Briefly, EIA plates (COSTAR®) were coated with 10 μg/ml of goat anti-human IgG Fc overnight at 4 C. After washing with 1×ELISA wash buffer (0.15 M NaCl, 0.02% Tween-20 (W/V)), the plates were incubated with about 50 μl of a 1:5 dilution of the 96-well supernatant for one hour at room temperature. After washing the plates with 1×ELISA wash buffer, alkaline phosphatase-conjugated goat anti-human IgG (heavy and light chains) (Jackson 109-055-088), and its substrate (Sigma® Aldrich 104-105), were used to detect the human IgG bound to the anti-Fc antibody coated on the plate.

Approximately one third of the colonies tested, i.e., the highest producers, were transferred to 24-well plates for further quantification and comparison of their expression levels. Cells were maintained in IMDM, 5% FBS, 2 mm glutamine and 1×MHX. Supernatants from spent 24-well cultures were assayed by ELISA as described above. The highest producing parental clones (primary transfectants) were identified based on the titers in 24-well spent cultures.

The seven top-producing. clones were subcloned to identify a higher-producing, more homogeneous cell line. Ninety-six-well tissue culture dishes were seeded at 5 cells/ml and 20 cells/ml in IMDM, 5% FBS, 2 mM glutamine and 1×MHX. The cells were incubated for about 14 days until colonies were visible. Cell supernatants from wells in which there was a single colony growing were assayed by ELISA, as described above. The higher-producing colonies were transferred to 24-well tissue culture dishes and the supernatants from spent cultures were assayed by ELISA. Eight clones were identified as the highest producers and these were subjected to a second round of subcloning in a manner identical to how the highest-producing first-round subclones wer identified.

Table 6 shows the antibody production titers for selected cell lines. Titers represent the value determined by ELISA on spent 24-well supernatant in IMDM, 5% FBS. Significant improvement in titers was not observed in the first round of subclones as compared to the parents, except for the subclone of parental clone 1 that doubled in IgG titer. The second round of subcloning did not yield any substantial increase in titer. Six of the highest-producing second-round subclones were selected for further characterization. Accordingly, the six cultures were assigned clone numbers for easy tracking. Table 6 shows the tracking designations and cell line codes of the six second-round subclones chosen for further characterization.

TABLE 6
Summary of Selected Production Cell
Lines and Antibody Titers.

| | | First- | Second- | | |
|---|---|---|---|---|---|
| | | Round | Round | | Cell |
| | | Subclone | Subclone | | Line |
| Parental | Titer | Titer | Titer | Tracking | (“C”) |
| Designation | (μg/ml) | (μg/ml) | (μg/ml) | Designation | Code |
| 1 | 25/30 | 60/50 | 43/55 | Clone #1 | C524A |
| 2 | 27/23 | 34/26 | 26/30 | Clone #2 | N/A |
| 3 | 20/16 | 30/30 | 24/30 | Clone #3 | N/A |
| 4 | 20/16 | 12/19 | 22/28 | Clone #4 | N/A |
| 5 | 60/40 | 24/34 | 35/28 | Clone #5 | C525A |
| 6 | 40/37 | 28/23 | 28/30 | Clone #6 | C526A |
| 7 | 60/40 | 25/38 | N/A | N/A | N/A |
| 8 | 20/16 | 23/24 | N/A | N/A | N/A |

Cell Line Development in Chemically Undefined Media and Chemically Defined Media

The following types of media were used in connection with the development of the C463A-derived, rTNV148B-producing cell line designated C524A:

1. SFM8 media: A chemically undefined medium. This serum-free but not protein-free medium comprises IMDM, Primatone® (Sheffield Prods., Hoffman Estates, Ill.), Albumin, and Excyte® (Bayer, Kankakee, Ill.).

2. IMDM, 5% FBS medium (optimal growth medium): A chemically undefined medium IMDM is available from, e.g., JRH Biosci. (Lenexa, Kans.), Cat. 51471. Fetal Bovine Serum is available from, e.g., Intergen Co. (Purchase, N.Y.), Cat. 1020-01, or HyClone (Logan, Utah), Cat. SH30071.

3. CDM medium: This CD medium is derived from SFM8 medium. CDM medium does not contain Primatone®, albumin, or Excyte®, all of which are present in SFM8 medium. CDM medium (Primatone®, albumin and Excyte® deprived SFM8 medium) is then supplemented with a 2× final concentration of trace elements A (Mediatech, Herdon, Va., Cat. 99 182-C1 , 1000× stock), a 2× final concentration of trace elements B (Mediatech, Cat. 99-175-C1, 1000× stock), a 2× final concentration of trace elements C (Mediatech, Cat. 99-176-C1, 1000× stock) and a 1× final concentration of vitamins (Mediatech, Cat. 25-020-C1, 100× stock) to make the complete CDM medium. The trace elements and vitamins do not contain components of animal origin.

4. CD-Hybridoma medium: a CD medium produced by Invitrogen, Carlsbad, Calif. (Cat.1 1279-023). CD-Hybridoma medium was supplemented with 1 g/L of NaHCO₃, and L-Glutamine to final concentrations of 6 mM.

Growth profiles and antibody titers of the transformed cell lines were compared to that of cell line C466D. C466D is another rTNV148B production cell line that is derived from mouse myeloma cells. C466D cells produce about 30 μg/ml IgG in IMDM, 5% FBS at T-flask and spinner flask scales.

The six selected cultures were expanded in IMDM, 5% FBS. Two to three vials from each cell line were frozen as safe freezes before weaning into CD media. During the process of expansion and weaning, some T-flask cultures from each cell line were set aside to overgrow until completely spent (12-14 days). IgG titers were determined by Nephlometry to evaluate each clone's capability to produce IgG.

Table 7 shows the IgG titers present in spent cultures from the six second-round subclones in various media at early stages of development. Based on IgG titers, Clones #2 through #4 were terminated from further development. The three remaining clones each produced over 100 μg/ml IgG in SFM8 medium. In IMDM, 5%FBS, however, only Clone #1 produced 90-100 μg/ml IgG compared to 30 μg/ml produced by C466D. Accordingly, C-code numbers C524A, C525A and C526A were assigned to Clone #1, Clone #5 and Clone #6, respectively, and a research cell bank (RCB) was made in IMDM, 5%FBS for each cell line.

TABLE 7
Doubling Time and IgG Titer of Subclones

| | IMDM, 5% FBS | | CD-Hyrbidoma | | SFM8 | |
|---|---|---|---|---|---|---|
| | Doubling | | Doubling | | Doubling | Titer |
| Clone | Time | Titer | Time | Titer | Time | (μg/ |
| Number | (hrs) | (μg/ml) | (hrs.) | (μg/ml) | (hrs.) | ml) |
| Clone #1 | 30-50 | 90-100 | 25-35 | 90-103 | 30-32 | 180 |
| Clone #5 | 25-28 | 45 | 35-40 | 68 | 20-25 | 130 |
| Clone #6 | 22-30 | 40 | 35-40 | 70 | 19-20 | 142 |
| Clone #2 | N/A | 40 | N/A | N/A | N/A | 63 |
| Clone #3 | N/A | 60 | N/A | N/A | N/A | 45 |
| Clone #4 | N/A | 50 | N/A | N/A | N/A | 57 |
| C466D | 25-30 | 30 | N/A | N/A | N/A | N/A |

The transfer of C466D cells into CD-Hybridoma medium failed in several attempts. The culture failed soon after cells were washed and transferred from IMDM, 5% FBS to CD-Hybridoma medium. However, C524A, C525A and C526A cells showed no difficulty in growing in CD-Hybridoma medium and were quickly expanded to spinner flasks to make a RCB from C524A and C526A. The approximate doubling times and overgrown IgG titers of CD-Hybridoma cultures of C524A, C525A and C526A are shown above in Table 7.

To follow up the observation that C524A produced nearly 100 μg/ml IgG in IMDM, 5% FBS and CD-Hybridoma medium, batch culture type growth profiles were performed to compare these two cultures to C466D grown in IMDM, 5% FBS. Duplicate cultures in 250 ml spinner flasks were seeded at a cell density of 2×10⁵ vc/ml in IMDM, 5% FBS and 3×10⁵ vc/ml in CD-Hybridoma medium. Each spinner flask contained 150 ml of medium and spinner speed was set at 60 rpm. One 2.5-ml sample was collected from each spinner flask for daily cell counts and IgG titer. Cultures were terminated after viability dropped below twenty percent.

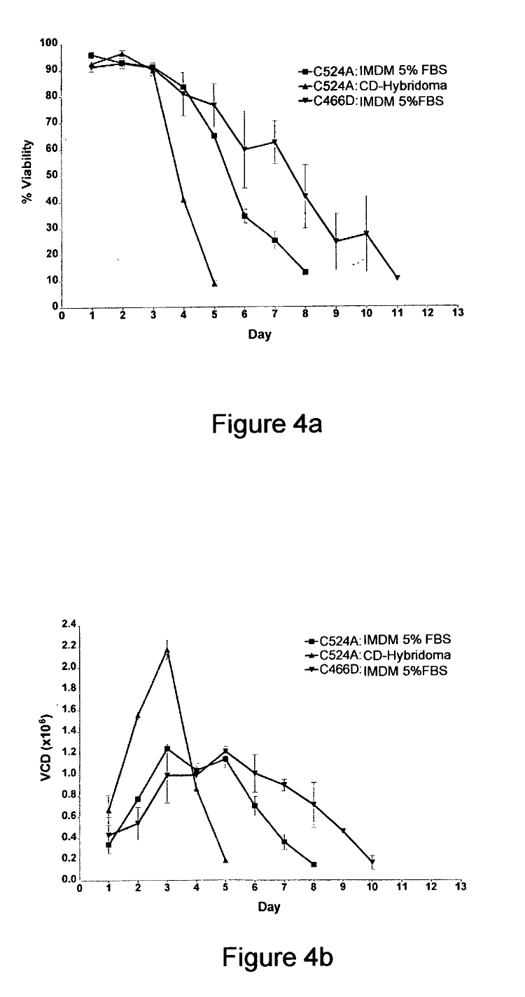

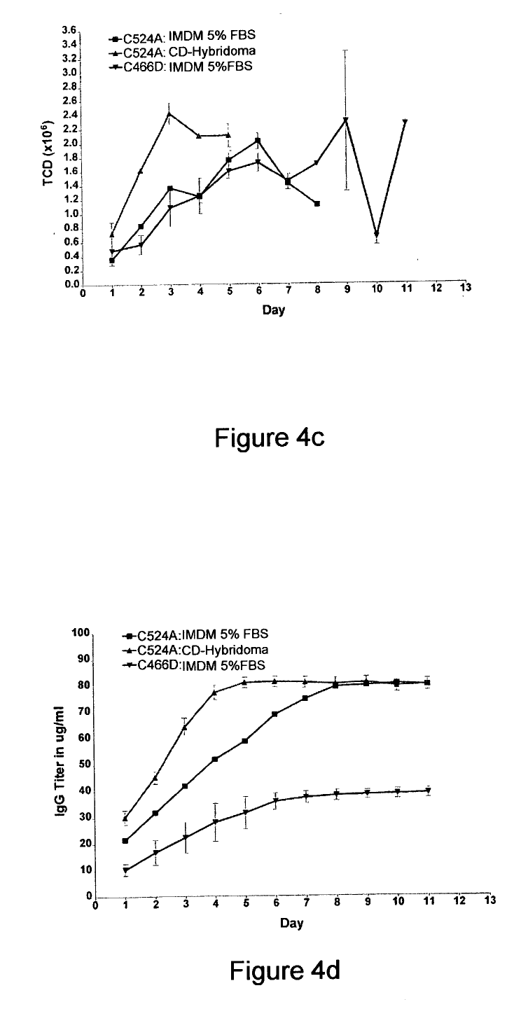

The data illustrated in FIG. 4 indicate that C524A cultures grown in either CD-Hybridoma medium or IMDM, 5% FBS grew at least as well as C466D grown in IMDM, 5% FBS. The total cell densities for all three cultures ranged from 2.2×10⁶ cells/ml to 2.4×10⁶ cells/ml (FIG. 4c), and total viable cell density ranged from 1.2×10⁶ cells/ml (both C524A and C466D in IMDM, 5% FBS) to 2.2×10⁶ cells/ml (C524A in CD-Hybridoma medium) (FIG. 4b). C524A in IMDM, 5% FBS lasted longer than the other two, based on the days that viability stayed above twenty percent (FIG. 4a). The final IgG titer of C524A in either CD-Hybridoma medium or IMDM, 5% FBS was around 80 μg/ml, compared to 30 μg/ml produced by C466D in IMDM, 5% FBS. The results indicate that C524A is a better rTNV 148B producing cell line than C466D.

The transfer of C524A, C525A and C526A into CDM medium was more difficult than the transfer into CD-Hybridoma medium (C466D failed to transfer into CDM medium). The cells did not grow for the first 2-3 passages and viability dropped to about forty percent or less. The surviving cells were then harvested and seeded into IMDM, 5% FBS for a few passages until viability was restored to about ninety percent. The rescued cells were then washed and seeded into CDM medium again. In most cases, this selection-rescue-selection process was repeated two to three times before cultures with good viability (>80%) and 30 to 40 hour doubling times were obtained. IgG titers of C525A and C526A in CDM medium were only about 60-70 μg/ml compared to 130 μg/ml produced by C524A in the same medium. Further characterization of C524A, C525A, and C526A revealed C524A to be the superior production cell line.

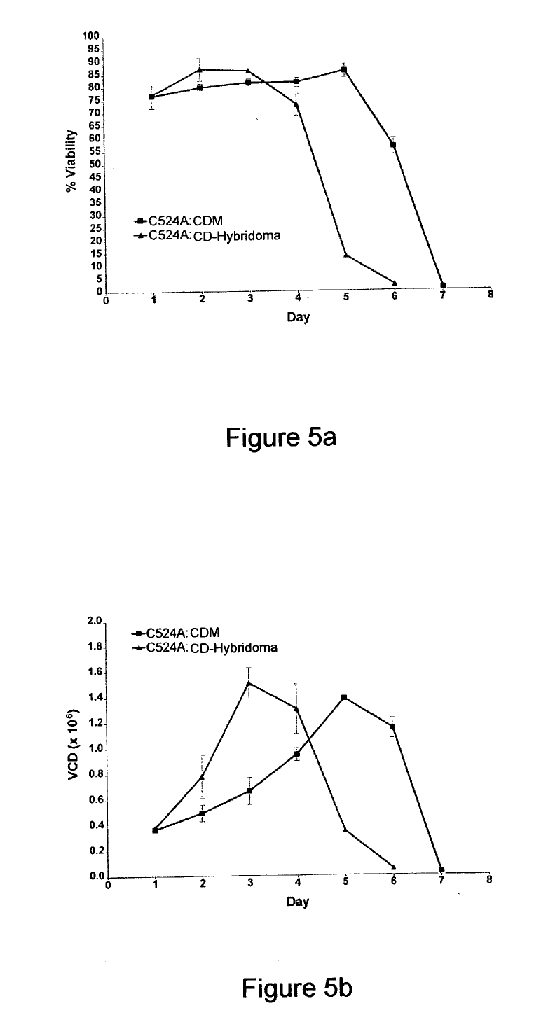

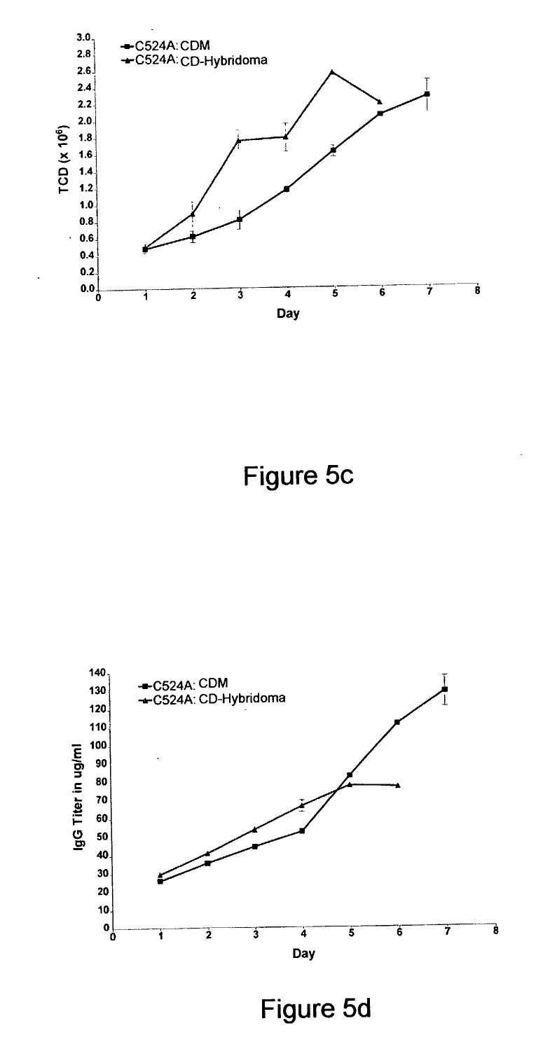

Utilizing the growth profile protocol described above, growth profiles of C524A in CD-Hybridoma medium and CDM medium were constructed to confirm the high IgG production phenotype in CDM medium. FIG. 5 shows that C524A cells grew faster in CD-Hybridoma medium than in CDM medium (FIG. 5a). These cells produced only about 70 μg/ml of IgG in CD-Hybridoma medium, compared to 130 μg/ml that C524A produced in CDM medium (FIG. 5d). C524A cultures in both media eventually reached the same total cell density and total viable cell density (FIG. 5b, 5c).

After RCBs were made, a ten-passage stability study was performed to examine the stability of cell growth and IgG production of C524A in CD-Hybridoma medium and CDM medium. One frozen vial from each RCB was thawed and expanded in either CD-Hybridoma medium or CDM medium to seed duplicate spinner flasks. Duplicate cultures in spinner flasks at 60 rpm were passaged every 2-3 days for 10 passages with a seeding density of 3×10⁵ vc/ml. Every week, triplicate T-25 flasks were set up from each spinner at 3×10⁵ vc/ml and allowed to overgrow for 7-8 days. The IgG titer for each week was determined as described above.

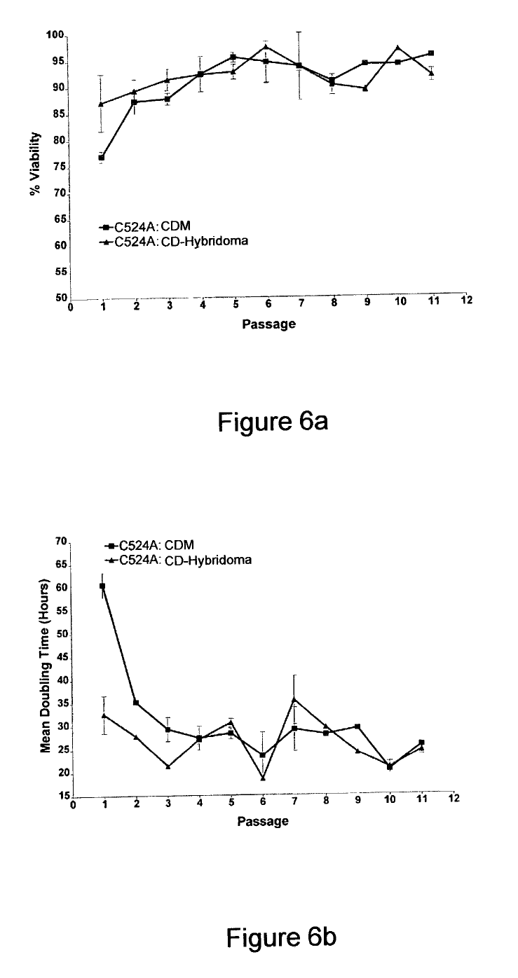

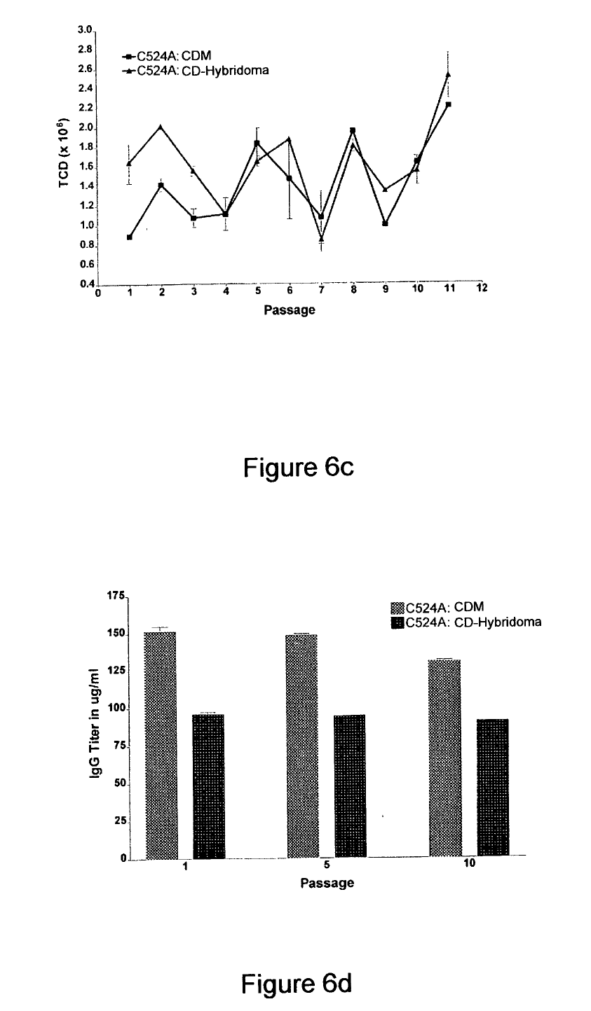

FIG. 6 shows that the doubling times of all four cell cultures (duplicate C524A cultures in CD-Hyrbidoma medium and CDM medium) ranged between 20-35 hours (FIG. 6b), and cell viabilities were consistently between eighty-five to ninety percent between passages 2 and 11 (FIG. 6a, 6b, 6c). IgG titer at the end of the stability study was eighty-three percent of the beginning culture for C524A in CDM medium, and was greater than ninety percent for C524A in CD-Hyrbidoma medium (FIG. 6d).

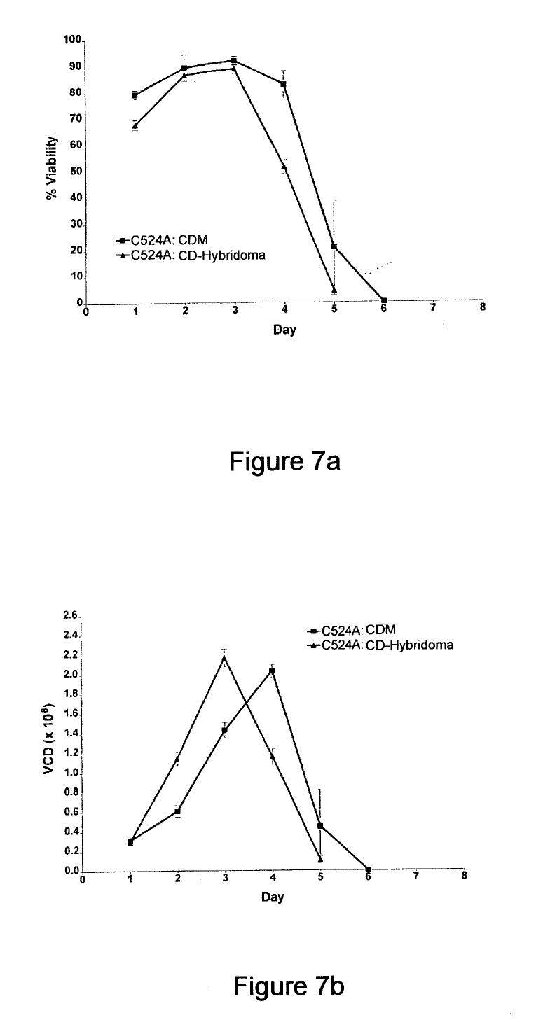

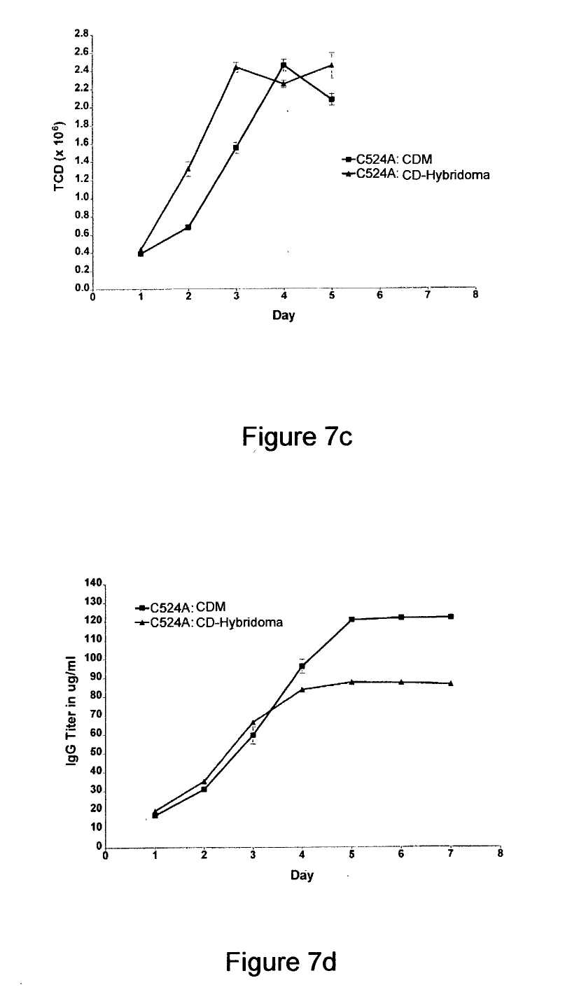

When these cultures reached passage 11, the cells were used to seed duplicate spinners for another growth profile. The cell growth of the second growth profile was slightly faster than the first profile performed at the beginning of ten-passage stability study (FIG. 7a, 7b and 7c). That result is similar to the one obtained in SFM8 medium (data not shown). In contrast to SFM8, there was a slight decrease (about 10%) in IgG titers. IgG titers of CDM cultures and CD-Hybridoma cultures were around 120 ug/ml and 80 ug/ml, respectively, in this growth profile study (FIG. 7d) compared to 130 μg/ml and 70 μg/ml from the previous growth profile study (FIG. 5d).

### Example 2

Transfection of C463A Cells in CD Media with Plasmids Encoding a Human Monoclonal Antibody (h-mAb).

h-mAb heavy chain expression vector is linearized by digestion with an appropriate restriction enzyme and h-mAb light chain expression vector is also linearized using an appropriate restriction enzyme. Prior to the transfection, C463A is thawed in a CD medium and grown for a few passages. Approximately 1×10⁷ C463A cells are transfected with about 10 μg of the premixed linearized plasmids by electroporation (200 V and 1180 μF). See Knight et al., 30 Molecular Immunology 1332 (1993). The transfection steps are all conducted using the same CD medium as the one used prior to transfection. Following transfection, the cells are seeded at a viable cell density of 1×10⁴ cells/well in 96-well tissue culture dishes with a CD medium. After incubating the cells at 37° C., 5% CO₂ for about 40 hours, an equal volume of a CD medium and 2×MHX selection is added. The plates are incubated at 37° C., 5% CO₂ for about two weeks until colonies become visible.

Cell supernatants from transfectant colonies are assayed after two weeks using the methods described in Examples 1 and 4. The clones producing the highest amount of IgG as determined by ELISA are transferred to 24-well plates containing a CD medium-and expanded for further quantification and comparison of IgG expression levels. Based on the amount of antibody produced, independent C463A transfectants are subcloned by seeding an average of one cell per well in 96-well plates. The quantity of antibody produced by the subclones is again determined by assaying supernatants from individual subclone colonies. Optimal subclones are selected for further analysis.

Growth curve analyses are performed on selected cell lines grown in CD media as described in Examples 1 and 4 and compared to the selected cell lines and control cell lines grown in optimal medium. In addition, stability studies of the selected cell lines grown in CD media are conducted as described in Examples 1 and 4 and compared to the selected cell lines and control cell lines grown in optimal medium.

The production of h-mAbs by the selected cell lines grown in a CD medium is comparable to antibody production by control cell lines either grown in optimal medium or transfected and maintained as in Example 1, in terms of quantity and quality. In addition, the selected cell lines grown in a CD medium are observed to stably produce h-mAbs at least as long as or longer than control cell lines.

### Example 3

Commercial-scale Culture of C524A for the Production of rTNV148B.

One vial of C524A cells is removed from liquid nitrogen, and thawed in a sterile 37° C. water bath. The cells are then removed, placed into sterile CD medium, and then expanded in spinner flasks at 37° C. After standard quality assays, and further expansion, cell cultures are pooled and introduced aseptically into a sterile, 500 liter or 1,000 liter bioreactor. A sterile CD medium is added to the bioreactor to the final desired volume, and the bioreactor system engaged for rTNV148B production. The bioreactor system is preferably a continous perfusion system, in which product-containing media is sieved by a spin filter, and harvested from the cell-containing retentate. Fresh sterile CD medium is replenished into the bioreactor to maintain nearly constant volume in the reactor vessel. Temperature, dissolved oxygen, pH, and cell density are monitored. Cell density and viability is observed throughout the production run, which is terminated when the cells have undergone the maximum doublings allowed by regulatory authorities, or when viability drops below twenty percent. The rTNV148B product may be purified by methods known in the art. Yield of rTNV148B averages from about 50 μg/ml to about 120 μg/ml.

### Example 4

Transfection of C463A Cells with Human Anti-IL-12 Monoclonal Antibody (hIL-12 mAb), to Produce the C463A-derived, hIL-12 mAb Production Cell Line.

Heavy chain expression vector is linearized by digestion with an appropriate restriction enzyme and light chain expression vector is also linearized using an appropriate restriction enzyme. C463A cells are transfected with about 10 μg of the premixed linearized plasmids by electroporation and cells cultured and transfectants selected as described in Example 1. Cell supernatants from transfectant colonies are assayed approximately two weeks later for human IgG (i.e., hIL-12 mAb). Briefly, cell supernatants are incubated on 96-well ELISA plates that are coated with goat antibodies specific for the Fc portion of human IgG. Human IgG bound to the coated plates is detected using alkaline phosphatase-conjugated goat anti-human IgG (heavy chain+light chain) antibody and alkaline phosphatase substrates as described.

Cells of the higher producing clones are transferred to 24-well culture dishes in standard medium and expanded (IMDM, 5% FBS, 2 mM glutamine, 1×MHX). The amount of antibody produced (i.e., secreted into the media of spent cultures) is carefully quantified by ELISA using purified hIL-12 mAb as the standard. Selected clones are then expanded in T-75 flasks and the production of human IgG by these clones is quantified by ELISA. Based on these values, independent C463A transfectants are subcloned (by seeding an average of one cell per well in 96-well plates), the quantity of antibody produced by the subclones is determined by assaying (ELISA) supernatants from individual subclone colonies. Optimal subclones, i.e., C463A transfectants, are selected for further analysis.

Assay for hIL-12 mAb Antigen Binding

Prior to subcloning the selected cell lines, cell supernatants from the parental lines are used to test the antigen binding characteristics of hIL-12 mAb. The concentrations of hIL-12 mAb in the cell supernatant samples are first determined by ELISA. Titrating amounts of the supernatant samples, or purified hIL-12 mAb positive control, are then incubated in 96-well plates coated with 2 μg/ml of human IL-12. Bound mAb is then detected with aLkaline phosphatase-conjugated goat anti-human IgG (heavy chain+light chain) antibody and the appropriate alkaline phosphatase substrates. hIL-12 mAb produced in C463A cells is preferably observed to bind specifically to human IL-12 in a manner indistinguishable from the purified hIL-12 mAb.

Characterization of Selected Cell Lines

Growth curve analyses are performed on selected cell lines by seeding T-75 flasks with a starting cell density of 2×10⁵ vc/ml in IMDM, 5% FBS or CD media. Cell number and hIL-12 mAb concentration are monitored on a daily basis until the cultures are spent. Sp_{2/0} parental cells transfected with hIL-12 mAb are grown in IMDM, 5% FBS as a control and growth curve analyses are performed. hIL-12 mAb production by the selected cell lines grown in a CD medium is preferably observed to be equal or superior to hIL-12 mAb production by Sp_{2/0} parental cells transfected with hIL-12 mAb and grown in optimal medium. Moreover, hIL-12 mAb production by the selected cell lines grown in a CD medium is preferably observed to be equal to or higher than hIL-12 mAb production by the selected cell lines grown in optimal growth medium.

The stability of hIL-12 mAb production over time for the selected cell lines is assessed by culturing cells in 24-well dishes with CD media or optimal growth medium for varying periods of time. The production of hIL-12 mAb by selected cell lines is also compared to production by Sp_{2/0} parental cells transfected with hIL-12 mAb and grown in optimal medium. hIL-12 mAb production by the selected cell lines grown in a CD medium is comparable to hIL-12 mAb production by Sp_{2/0} parental cells transfected with hIL-12 mAb and grown in optimal medium, in terms of quality and quantity. In addition, selected cell lines grown in a CD medium are stably produce hIL-12 mAb for a term comparable to that of Sp_{2/0} parental cells transfected with hIL-12 mAb and grown in optimal medium.

We claim:

1. A clonal myeloma cell line or any cell line derived capable of:
growing continuously in a chemically defined medium;
growing to high cell density in a chemically defined medium;
remaining viable after cryopreservation in the absence of serum; and
detectably expressing recombinant proteins following genetic manipulation and/or subsequent culture in a chemically defined medium.

2. The cell line of claim 1, wherein said expressing is accomplished by manipulating said cell line or cell line derived therefrom to express at least one protein in detectable amounts.

3. The cell line of claim 2, wherein said manipulation is selected from the group consisting of introducing a nucleic acid encoding at least one protein into said cell line, and inducing transcription and translation of a nucleic acid encoding at least one protein when such nucleic acid already exists in said cell line.

4. The cell line of claim 3, where said introducing step is selected from the group consisting of electroporation, lipofection, calcium phosphate precipitation, polyethylene glycol precipitation, sonication, transfection, transduction, transformation, and viral infection.

5. The cell line of claim 2, where said at least one protein is selected from the group consisting of a diagnostic protein and a therapeutic protein.

6. The cell line of claim 5, where said diagnostic or therapeutic protein is selected from one or more of the group consisting of an immunoglobulin, a cytokine, an integrin, an antigen, a growth factor, a cell cycle protein, a hormone, a neurotransmitter, a receptor or fusion protein thereof, a blood protein, an antimicrobial, any fragment thereof, and any structural or functional analog thereof.

7. The cell line of claim 6, wherein said immunoglobulin or fragment is selected from one or more of the group consisting of rodent, primate, chimeric, and engineered.

8. The cell line of claim 7, wherein said immunoglobulin or fragment is selected from one or more of the group consisting of murine, human, chimeric, humanized, CDR grafted, phage displayed, transgenic mouse-produced, optimized, mutagenized, randomized, and recombined.

9. The cell line of claim 8, wherein said immunoglobulin or fragment is selected from one or more of the group consisting of IgG1, IgG2, IgG3, IgG4, IgA 1, IgA2, slgA, IgD, IgE, and any structural or functional analog thereof.

10. The cell line of claim 8, wherein said fragment is selected from one or more of the group consisting of F(ab′)₂, Fab′, Fab, Fc, Facb, pFc′, Fd, Fv, and any structural or functional analog thereof.

11. The cell line of claim 8, wherein said immunoglobulin or fragment thereof binds one or more of the group consisting of an immunoglobulin, a cytokine, an integrin, an antigen, a growth factor, a cell cycle protein, a hormone, a neurotransmitter, a receptor or fusion protein thereof, a blood protein, an antimicrobial, any fragment thereof, and any structural or functional analog thereof.

12. The cell line of claim 6, wherein said integrin is selected from one or more of the group consisting of α1 , α2, α3, α4, α5, α6, α7, α8, α9, αD, αL, αM, αV, αX, αIIb, αIELb, β1, β2, β3, β4, β5, β6, β7, β8, α1β1, α2β1, α3β1, α4β1, α5β1, α6β1, α7β1, α8β1, α9β1, α4β7, α6β4, αDβ2, αLβ2, αMβ2, αVβ1, αVβ3, αVβ5, αVβ6, αVβ8, αXβ2, αIIbβ3, αIELbβ7, and any structural or functional analog thereof.

13. The cell line of claim 6, wherein said antigen is derived from one or more of the group consisting of a bacterium, a virus, a blood protein, a cancer cell marker, a prion, a fungus, and any structural or functional analog thereof.

14. The cell line of claim 6, wherein said growth factor is selected from one or more of the group consisting of a human growth factor, a platelet derived growth factor, an epidermal growth factor, a fibroblast growth factor, a nerve growth factor, a human chorionic gonadotropin, an erythrpoeitin, an activin, an inhibin, a bone morphogenic protein, a transforming growth factor, an insulin-like growth factor, and any structural or functional analog thereof.

15. The cell line of claim 6, wherein said cell cycle protein is selected from one or more of the group consisting of a cyclin, a cyclin-dependent kinase, a tumor suppressor gene, a caspase protein, a Bcl-2, a p70 S6 kinase, an anaphase-promoting complex, a S-phase promoting factor, a M-phase promoting factor, and any structural or functional analog thereof.

16. The cell line of claim 6, wherein said cytokine is selected from one or more of the group consisting of an interleukin, an interferon, a colony stimulating factor, a tumor necrosis factor, an adhesion molecule, an angiogenin, an annexin, a chemokine, and any structural or functional analog thereof.

17. The cell line of claim 6, wherein said hormone is selected from one or more of the group consisting of a human growth hormone, a growth hormone, a prolactin, a follicle stimulating hormone, a human chorionic gonadotrophin, a leuteinizing hormone, a thyroid stimulating hormone, a parathyroid hormone, an estrogen, a progesterone, a testosterone, an insulin, a proinsulin, and any structural or functional analog thereof.

18. The cell line of claim 6, wherein said neurotransmitter is selected from one or more of the group consisting of an endorphin, a coricotropin releasing hormone, an adrenocorticotropic hormone, a vaseopressin, a giractide, a N-acytlaspartylglutamate, a peptide neurotransmitters derived from pre-opiomelanocortin, any antagonists thereof, and any agonists thereof.

19. The cell line of claim 6, wherein said receptor or fusion protein thereof is selected from one or more of the group consisting of an interleukin-1, an interleukin-12, a tumor necrosis factor, an erythropoeitin, a tissue plasminogen activator, a thrombopoetin, and any structural or functional analog thereof.

20. The cell line of claim 6, wherein said blood protein is selected from one or more of the group consisting of an erythropoeitin, a thrombopoeitin, a tissue plasminogen activator, a fibrinogen, a hemoglobin, a transferrin, an albumin, a protein c, and any structural or functional analog thereof.

21. The cell line of claim 6, wherein said antimicrobial is selected from one or more the group consisting of a beta-lactam, an aminoglycoside, a polypeptide antibiotic, and any structural or functional analog thereof.

22. The cell line of claim 2, wherein said protein is produced at about 0.01 mg/L to about 10,000 mg/L of culture medium of said cell line.

23. The cell line of claim 2, wherein said protein is produced at a level of about 0.1 pg/cell/day to about 100 ng/cell/day.

24. A method for producing at least one protein from a cultured cell, comprising:
culturing cells of the cell line of claim 1 or 2 in a chemically defined medium, wherein said cells express said at least one desired protein; and
isolating said at least one desired protein from said chemically defined medium or said cells.

25. An isolated protein obtained from cells according to the method of claim 24.

26. A method for identifying cell lines capable of growing continuously in a chemically defined medium comprising the steps of:
culturing cells from one type of cell line in at least one type of chemically defined medium, wherein said cultured cells from one type of cell line are not known to grow in said chemically defined medium; and
selecting spontaneous mutant cells that are capable of growing in said chemically defined medium.

27. At least one cell line obtained according to the method of claim 26.

28. A protein obtained from the cell line of claim 1.

29. The method of doing business comprising the step of:
providing a customer with a cell line according to claim 1.

30. The method of doing business comprising the step of:
providing a customer with a protein derived from at least one cell line according to claim 1.

31. The cell line of claim 9, wherein said immunoglobulin is infliximab.

32. The cell line of claim 9, wherein said immunoglobulin is rTNV148B.

33. The cell line of claim 10, wherein said fragment is abciximab.

34. The cell line of claim 20, wherein said blood protein is tissue plasminogen activator.
